(19) United States
(12) Patent Application Publication
(10) Pub. No.: US 2006/0270909 A1
(43) Pub. Date: Nov. 30, 2006

(54) Surgical instruments and methods for use in reduced-access surgical sites

(76) Inventors: John W. Davis, Sunnyvale, CA (US); Geoffrey H. Willis, Redwood City, CA (US); Eric J. Willis, Santa Cruz, CA (US); Charles Gresl, San Francisco, CA (US); Peter Callas, Redwood City, CA (US); Thomas Vassiliades, Atlanta, GA (US)

Correspondence Address: LAW OFFICE OF ALAN W. CANNON, 834 SOUTH WOLFE ROAD, SUNNYVALE, CA, 94086, US

(21) Appl. No.: 11/137,255

(22) Filed: May 25, 2005

(51) Int. Cl. A61B 1/32

(52) U.S. Cl. 600/210

(57) ABSTRACT

Devices and methods for stabilizing a portion of an organ or tissue in a closed or restricted space surgical site. Devices and methods for manipulating an organ or tissue in a closed or restricted space surgical site. Devices and methods for positioning and orienting stabilizers and manipulators I a closed or restricted space surgical site.

### FIELD OF THE INVENTION

The present invention pertains to the field of surgical instruments and techniques, and more particularly to surgical instruments and techniques for performance in reduced or restricted working spaces.

### BACKGROUND OF THE INVENTION

Certain surgical procedures require the surgeon to perform delicate surgical operations on tissues within the body that are moving or otherwise unstable. The ability to stabilize or immobilize a surgical site provides greatly improved surgical accuracy and precision and reduces the time required to complete a particular procedure. A large and growing number of surgeons are performing successful coronary artery bypass graft (CABG) surgery on the beating heart by temporarily stabilizing or immobilizing a localized area of the beating heart. Methods and apparatus for performing a CABG procedure on a beating heart are described in U.S. Pat. Nos. 5,894,843 and 5,727,569 to Benetti et al., the disclosures of which are herein incorporated by reference.

In a typical CABG procedure, a blocked or restricted section of coronary artery, which normally supplies blood to some portion of the heart, is bypassed using a source vessel or graft vessel to re-establish blood flow to the artery downstream of the blockage. This procedure requires the surgeon to create a fluid connection, or anastomosis, between the source or graft vessel and an arteriotomy or incision in the coronary artery. Forming an anastomosis between two vessels in this manner is a particularly delicate procedure requiring the precise placement of tiny sutures in the tissue surrounding the arteriotomy in the coronary artery and the source or graft vessel.

The rigors of creating a surgical anastomosis between a coronary artery and a source vessel or graft vessel demands that the target site for the anastomosis be substantially motionless. To this end, a number of devices have been developed which are directed to stabilizing a target site on the beating heart for the purpose of completing a cardiac surgical procedure, such as completing an anastomosis. Stabilization may be provided using a device that provides a mechanical or compression force to the tissue or by a device which applies a negative pressure or suction to the tissue. Representative devices useful for stabilizing a beating heart are described, for example, in U.S. Pat. Nos. 5,894,843; 5,727,569; 5,836,311 and 5,865,730.

As beating heart procedures have evolved, regardless of whether compression or negative pressure has been used to stabilize or immobilize the heart, new challenges have arisen. For example, surgeons may gain access to the heart using a number of different approaches, both open and closed chest, such as through a sternotomy, mini-sternotomy, thoracotomy or mini-thoracotomy, or less invasively through a port provided within the chest cavity of the patient, e.g., between the ribs or in a subxyphoid area, with or without the visual assistance of a thoracoscope. Accordingly, the devices used to stabilize the heart must be configured to accommodate the particular approach chosen. For example, when a closed chest approach is used such as a port access approach wherein the device is introduced into the body cavity through a small access port or stab wound, the device must be designed to be advanced through such small openings without damaging the device or any internal body structures. A continuing need remains for new and better instruments that are capable of being delivered through small openings and still function satisfactorily in a closed-chest environment.

Furthermore, in addition to addressing delivery problems of instruments though small access openings, the working space within a closed-chest surgical environment is extremely limited, allowing much less room to maneuver the instruments, as compared to the space provided in an open-chest surgical site, once they have been successfully delivered or placed in the operative site. Thus, new and better approaches, tools and techniques for controlling instruments in a closed chest environment are needed.

As such, there is continued interest in the development of new devices and methods for easily and effectively stabilizing or immobilizing tissue, e.g., a beating heart, in a limited space environment, such as occurs during closed-chest procedures. Of particular interest would be the development of such devices and methods of use which may be used in a variety of surgical approaches, including a sternotomy, mini-sternotomy, thoracotomy, mini-thoracotomy, transabdominal, and particularly in less invasive techniques such as endoscopic or port access procedures (e.g., between the ribs or in a subxyphoid area), with or without the visual assistance of a thoracoscope.

### SUMMARY OF THE INVENTION

The present invention provides a stabilizer assembly for stabilizing a portion of an organ in a closed or restricted space surgical site, wherein the assembly includes: a stabilizer foot adapted to be delivered to a location of the organ through a first opening in a patient and adapted to contact a surface of the organ to provide stabilization thereto during a surgical procedure; and a support arm adapted to be delivered to the location of the organ through a second opening in the patient smaller than the first opening. The stabilizer foot includes at least one connecting member and the support arm includes a connecting feature at a distal end thereof, adapted to connect the support arm to the stabilizer foot via one of the connecting members. The connection may be accomplished in the closed or restricted space.

In another aspect of the present invention, a stabilizer foot is provided for contacting an organ to stabilize a portion thereof in a closed or restricted space surgical site. The stabilizer foot includes at least one contact surface formed on an inferior side thereof which is adapted to contact a surface of the organ. At least one grab member is provided on a superior side of the stabilizer foot that is adapted to be grabbed by a grasping tool, wherein the stabilizer foot is configured to be inserted through an opening in a patient to contact the surface of the organ, and wherein the at least one grab member is adapted to be grasped by extending the grasping tool through the opening, while controlling the grasping tool from outside the opening.

A stabilizer assembly is provided for stabilizing a portion of an organ in a closed or restricted space surgical site, the assembly including a stabilizer foot adapted to be delivered to a location of the organ through an opening in a patient and adapted to contact a surface of the organ to provide stabilization thereto during a surgical procedure. The stabilizer foot has at least one opening in a contact surface to apply negative pressure to a surface of the organ, and at least one vacuum line fluidly connected to the at least one contact surface opening and adapted to connect with a vacuum source external of the surgical site. Soft tissue retracting tapes are attached to the stabilizer foot and are adapted to be fixed externally of the opening in the patient after connection of the stabilizer foot to the organ via suction to stabilize a portion of the organ.

A stabilizer foot and positioner assembly are provided for use in stabilizing a portion of an organ in a closed or restricted space surgical site. The assembly includes a stabilizer foot adapted to be delivered to a location of the organ through a first opening in a patient and adapted to contact a surface of the organ to provide stabilization thereto during a surgical procedure; and an elongated positioner extending from the stabilizer foot and having sufficient length to deliver the stabilizer foot to the surface of the organ by manipulating the positioner from outside the first opening.

A support arm adapted to connect with a stabilizer foot in a closed or restricted space surgical site, wherein the stabilizer foot has been inserted through a first opening in a patient and the support arm is inserted through a second opening in the patient is provided. The support arm includes an elongated body adapted to pass through the second opening and having sufficient length to extend out of the second opening after connecting the support arm with the stabilizer foot; a connecting feature disposed at a distal end of the elongated body; and means for moving the connecting feature between a disconnected configuration in which the connecting feature may be readily disconnected from the stabilizer foot, and a connected configuration, in which the connecting feature securely connects with the stabilizer foot; wherein the means for moving is positioned for manipulation outside of the patient by a user.

A manipulator assembly for moving or positioning an organ in a closed or restricted space surgical site is provided, including a low profile suction member adapted to be delivered to a location of the organ through a first opening in a patient and adapted to contact a surface of the organ and to attach to the organ using vacuum. The suction member further includes at least one grab member on a superior surface thereof. The said grab member(s) is/are configured to be engaged by a tool for applying force thereto to reposition the suction member on the organ. A suction line extends from the suction member and has sufficient length to extend out of the first opening, or a second opening in the patient for connection with an external vacuum source.

A manipulator assembly for moving or positioning an organ in a closed or restricted space surgical site is provided to include a suction member adapted to be delivered to a location of the organ through a first opening in a patient and adapted to contact a surface of the organ and to attach to the organ using vacuum; and a positioner connected to the suction member. The positioner is flexible in bending and torsionally stiff, and is adapted to be drawn through the first opening and rerouted through a second opening in a patient. The positioner has sufficient length to extend proximally out of the second opening when the suction member is attached to a surface of the organ at the desired location.

A manipulator assembly for moving or positioning an organ in a closed or restricted space surgical site is provided, including a suction member adapted to be delivered to a location of the organ through a first opening in a patient and adapted to contact a surface of the organ and to attach to the organ using vacuum; and a positioner connected to the suction member. The positioner is flexible in bending and torsionally stiff, and has sufficient length to extend proximally out of the patient when the suction member is attached to a surface of the organ at a desired location.

A manipulator assembly for moving or positioning an organ in a closed or restricted space surgical site is provided that includes a low-profile suction member adapted to be delivered to a location of the organ through a first opening in a patient and adapted to contact a surface of the organ and to attach to the organ using vacuum; and a rigid positioner connected to the suction member. The positioner has sufficient length to extend proximally out of the patient when the suction member is attached to a surface of the organ at a desired location.

A suction manipulator is provided, including a suction member adapted to be delivered to a location of an organ through a first opening in a patient and adapted to contact a surface of the organ and to attach to the organ using vacuum. The suction member includes at least one grab member on a superior surface thereof. Each grab member is configured to be engaged by a tool for applying force thereto to reposition the suction member on the organ.

An extremely low profile manipulator is provided to include a main body formed of a flexible membrane; at least one grab member on a superior surface of the main body, configured to be engaged by a tool for applying force thereto to reposition the main body on an organ; and means for attaching the main body to the organ.

A method of stabilizing a portion of an organ in a closed or restricted space surgical site includes the steps of: delivering a stabilizer foot through a first opening in a patient and contacting a surface of the organ with the stabilizer foot; inserting a distal end of a support arm through a second opening in the patient while controlling the distal end from a proximal end portion of the support arm outside the patient; and connecting the distal end of the support arm to the stabilizer foot.

In another aspect of the invention, a method of stabilizing a portion of an organ in a closed or restricted space surgical site includes the steps of: delivering a stabilizer foot through a first opening in a patient and contacting a surface of the organ with the stabilizer foot; applying negative pressure between the stabilizer foot and the organ to fix the stabilizer foot to the organ; applying tension to soft tissue retractor tapes attached to the stabilizer foot, from a location outside of the first opening, to stabilize a portion of the organ; and fixing the soft tissue retractor tapes to a relatively stationary object to maintain the applied tension and stabilization.

A method of positioning a stabilizer on a portion of an organ of a patient in a closed or restricted space surgical site is provided, including the steps of: delivering a stabilizer foot through a first opening in a patient and contacting a surface of the organ with the stabilizer foot; grasping a portion of the stabilizer foot using at least one grasper operated from outside the patient and applying force to the stabilizer foot via the grasper to perform at least one of relocating and reorienting the stabilizer foot; and removing all graspers from the restricted space surgical site when a desired orientation and location of the stabilizer foot has been achieved.

A method of manipulating an organ or tissue in a closed or restricted space surgical site is provided, including the steps of: delivering a suction member through a first opening in a patient and contacting a surface of the organ or tissue with the suction member; positioning the suction member in a desired location on the organ or tissue by applying force to the suction member via at least one element extending out of the patient, from a location outside of the patient; delivering vacuum to the suction member when the suction member has been positioned in the desired location, to establish negative pressure between the suction member and the surface of the organ or tissue such that the suction member attaches to the organ or tissue; and exerting force on the suction member, via at least one element extending out of the patient, sufficient to move the suction member and the attached organ or tissue.

A method of positioning a manipulator on a portion of an organ or tissue of a patient in a closed or restricted space surgical site is disclosed to include the steps of: delivering a manipulator head through a first opening in a patient and contacting a surface of the organ or tissue with the manipulator head; grasping a portion of the manipulator head using at least one grasper operated from outside the patient and applying force to the manipulator head via the grasper to perform at least one of relocating and reorienting the manipulator head; removing all graspers from the restricted space surgical site when a desired orientation and location of the manipulator head has been achieved; and attaching the manipulator head to the surface of the organ or tissue.

A method of manipulating an organ or tissue of a patient in a closed or restricted space surgical site as disclosed includes the steps of: delivering a manipulator head through a first opening in a patient and contacting a surface of the organ or tissue with the manipulator head; positioning the manipulator head in a desired location on the organ or tissue by grasping a portion of the manipulator head using at least one grasper operated from outside the patient and applying force to the manipulator head via the grasper to perform at least one of relocating and reorienting the manipulator head; attaching the manipulator head to the surface of the organ or tissue; and moving the organ or tissue or maintaining the organ or tissue in a displaced location by applying force to at least one portion of the manipulation head via at least one grasper, from at least one location outside of the patient.

These and other advantages and features of the invention will become apparent to those persons skilled in the art upon reading the details of the devices, assemblies and methods as more fully described below.

### BRIEF DESCRIPTION OF THE DRAWINGS

FIG. 1 shows a cutaway view showing use of a stabilizer in the performance of a closed-chest, beating heart, coronary artery bypass graft (CABG) procedure according to the present invention.

FIG. 2A shows a partial view of support arm including jaws for attachment according to the present invention.

2B shows a partial, sectional view illustrating one example of a mechanism for operating the jaws shown in FIG. 2A for locking them to a ball member.

FIG. 2C shows an example of jaws provided with features that assist the lead in of a ball member within the confines of the open jaws.

FIG. 2D shows an example of jaws which are joined at their proximal ends.

FIG. 2E shows an example of jaws which are separate.

FIGS. 2F, 2G and 2H show other examples of jaw arrangements.

FIG. 3A shows another example of stabilizer foot configured with a removable positioning member.

FIG. 3B shows the stabilizer foot of FIG. 3A after contacting the positioning member therewith.

FIG. 3C shows an example of a mechanism for securing the position of the positioning member relative to the stabilizer foot.

FIG. 3D shows an example of a positioning member that is directly connected to a stabilizer foot, without the use of an intermediate joint such as a ball joint.

FIG. 4 shows a stabilizer assembly having alternative examples of locking mechanisms for both the support arm and the positioning member.

FIG. 5 shows an example of a stabilizer foot that is configured for positioning without the use of a positioning member.

FIG. 6 shows a variation in a manner in which a support arm may be connected with a stabilizer foot.

FIG. 7 shows another variation for connecting a support arm with a stabilizer foot 12.

FIG. 8A shows still another variation for connecting a support arm with a stabilizer foot.

FIG. 8B illustrates a locking mechanism that may be employed with the variation of FIG. 8A.

FIG. 9A shows a variation of an arrangement at the distal end of a support arm for making a connection with a ball member.

FIG. 9B shows the support arm of FIG. 9A and associated driver assembly for locking the support arm to a stabilizer foot.

FIG. 10 illustrates a principle for design of ball and socket type connections that are provided in connections, particularly between a support arm and a stabilizer foot, or between a positioning member and a stabilizer foot.

FIG. 11A shows a modified arrangement of a support arm employing jaws for connecting with a stabilizer foot.

FIG. 11B is a partial view showing a mechanism for operating the jaws of the arrangement shown in FIG. 11A.

FIG. 12A shows an example of a stabilizer foot having at least a stem formed from a malleable material.

FIG. 12B shows an example of a stabilizer foot provided with multiple connection members attached to a sheet metal layer which is malleable.

FIG. 12C shows an example of a stabilizer foot having multiple connection members interconnected by wire form which may be of the same malleable material that the connection members are made of.

FIG. 12D shows a variation of the arrangement of FIG. 12B in which sheet metal appendages or extensions extend from the sheet metal layer to provide malleability for re-orienting the connection members.

FIG. 13A illustrates another arrangement for connecting a support arm to a stabilizer foot, in which a socket member is affixed to the stabilizer foot and the distal end of the support arm includes a connection member comprising a ball member and stem.

FIG. 13B shows a variation of the ball expansion mechanism in FIG. 13A, in which the ball member is a solid, split ball that is expandable by drawing a wedge or cam member to further split and expand the ball.

FIG. 14 is a partial view of a stabilizer assembly which includes a mechanism for magnetically coupling a support arm to a stabilizer foot.

FIG. 15 shows an example of a stabilizer assembly in which one or more vacuum lines that are used to supply negative pressure to a vacuum-type stabilizer foot are also used to guide and align the connecting mechanism for joining a support arm with a stabilizer foot.

FIG. 16 shows a stabilizer assembly that does not require a support arm in order to stabilize a portion of the organ to which it is attached.

FIG. 17A shows an organ manipulator for use in closed-chest or limited space surgical sites according to the present invention.

FIG. 17B is a side view of a suction member that may be employed in a manipulator assembly according to the present invention.

FIG. 17C is a partial, bottom view of the manipulator assembly of FIG. 17A.

FIG. 17D shows a manipulator assembly in which grab members comprise nubs extending from locations distributed over the superior surface of a suction member.

FIG. 17E is a side view of a suction member showing a variation of grab members.

FIG. 17F is a partial top view of a manipulator assembly showing another variation of grab member.

FIG. 17G shows another example of a manipulator assembly provided with grab members.

FIG. 17H shows still another example of a manipulator assembly provided with grab members.

FIG. 18 shows a manipulator assembly in which a positioning member is used to engage grab members on a suction member to position and reorient it.

FIG. 19A shows another example of a manipulator assembly according to the present invention.

FIG. 19B shows a malleable stylet that is used to define curvature in the tubular shaft of the assembly of FIG. 19A.

FIG. 19C shows an assembly formed by the manipulator of FIG. 19A and the stylet of FIG. 19B in performing manipulation of an organ.

FIG. 19D is a partially cutaway view of an example of a tubular shaft component that may be employed in the manipulator of FIG. 19C.

FIG. 19E is a partially cutaway view of another example of a tubular shaft component that may be employed in the manipulator of FIG. 19C

FIG. 19F is an exploded view illustrating components use in operating the manipulator of FIG. 19C.

FIG. 20A shows another example of a manipulator that employs a rigid positioner.

FIG. 20B shows a variation of the manipulator shown in FIG. 20A.

FIG. 20C shows a sectional view of another manipulator that includes a suction member similar to the manipulator of FIG. 20A, but which does not include an integrated rigid positioner.

FIG. 20D shows a manipulator with a sectional view of a low profile suction member.

FIG. 20E is a perspective view of another low profile manipulator in accordance with the present invention.

FIG. 20F shows a planar view and FIG. 20G shows a side view of an extremely low profile manipulator according to the present invention.

### DETAILED DESCRIPTION OF THE INVENTION

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a ball” or “a ball joint” includes a plurality of such balls or ball joints and reference to “the contact member” includes reference to one or more contact members and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The term “open-chest procedure” refers to a surgical procedure wherein access for performing the procedure is provided by a full sternotomy, wherein the sternum is incised and the patient's ribs are separated using a sternal retractor to open the chest cavity for access thereto.

The term “closed-chest procedure” refers to a surgical procedure wherein access for performing the procedure is provided by one or more openings which are much smaller than the opening provided by an open-chest procedure, and wherein a traditional sternotomy is not performed. Closed-chest procedures may include those where access is provided by any of a number of different approaches, including mini-sternotomy, thoracotomy or mini-thoracotomy, or less invasively through a port provided within the chest cavity of the patient, e.g., between the ribs or in a subxyphoid area, with or without the visual assistance of a thoracoscope.

As alluded to above, surgical procedures carried out on a patient by “closed-chest” procedures allow much less free space for the surgeon to work in than do more conventional “open-chest” procedures such as those where access is gained through a sternotomy, for example. As such, the instruments used during closed-chest procedures must be as non-obstructive as possible and require only minimal amounts of space for their use. The present invention provides devices and methods that are useful for performing surgical procedures where working space is limited.

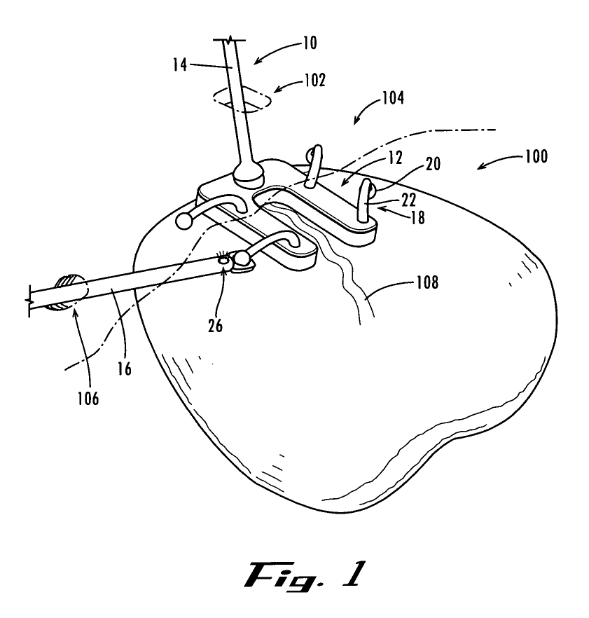

FIG. 1 shows a cutaway view 100 showing use of a stabilizer 10 in the performance of a closed-chest, beating heart, coronary artery bypass graft (CABG) procedure. In this procedure, a primary thoracotomy 102 has been formed in the patient's chest 104, of a size large enough to pass stabilizer foot 12 therethrough, and to allow sufficient space for the surgeon to work through in performance of dissection and anastomosis procedures. However, a primary opening other than a thoracotomy may alternatively be employed, such as a mini-sternotomy, sub-sternal opening, or the like. To assist in providing as much working space as possible through primary opening/thoracotomy 102, positioning member 14 is made relatively slender and preferably has a tapering cross-section size from where it connects with stabilizer foot 12, as it extends away from stabilizer foot 12. Although positioning member 14 is shown integral with stabilizer foot 12 in FIG. 1, it may also be configured to be removable from stabilizer foot 12, once the desired positioning of stabilizer foot 12 has been accomplished, thereby increasing the working space available through thoracotomy 102.

Stabilizer foot 12 is adapted to contact the heart or other organ or tissue to provide stabilization thereto during a surgical procedure. Stabilizer foot 12 may include a pair of foot members or contact members as shown in FIG. 1, which may be connected to form a substantial U-shape, and which may be substantially planar, or slightly curved to conform to the shape of the heart or other organ or tissue, or one or more contact members may have a non-conforming curve to establish a contact between only a portion thereof and the beating heart or other organ and tissue. Stabilizer foot 12 may be malleable such that the shape of the contact members may be varied depending on the clinical assessment by the surgeon, the design of the remainder of the stabilizer 10, and/or the design of other instruments to be used to complete the anastomosis.

Stabilizer foot 12 may effect stabilization by application of negative pressure to the surface to be stabilized, which draws the surface against the contact members of stabilizer foot 12 when the negative pressure is applied. Alternatively, stabilizer foot 12 may be a mechanical stabilizer that effects stabilization through the application of physical pressure against the surface contacted. Further, a combination of negative pressure (suction) and physical (mechanical) pressure against the surface to be stabilized is possible. Thus, alternative stabilizer feet 12 may be utilized to offer a choice of a different design or configuration, or even principle of operation. For example a change from a mechanical stabilizer foot 12, which operates by applying physical pressure against the beating heart tissue, may be replaced with a negative pressure stabilizer foot 12, which engages the heart by vacuum. In this regard, any of the stabilizer feet 12 described herein could be exchanged for operation in the stabilizer 10 described.

Additionally, other known contact members/stabilizer feet could be used or adapted to be used by those of ordinary skill in the art. The contact members of stabilizer foot 12 may have frictional surfaces on the underside thereof to more securely engage the tissue that they contact. The tips or ends of the contact members may be bent upward in the forms of “ski tips” to prevent edge effects (e.g., stress concentration, cutting, chafing, etc.) against the tissue which might otherwise be caused by straight tips. The contact members may also be modified to include apertures, openings or attachments to facilitate connection with sutures or other devices used to achieve the stabilization and/or anastomosis. Examples of alternative contact members/stabilizer feet that may also be modified for use with the present stabilizer can be found, for example, in U.S. Pat. Nos. 6,036,641; 6,283,912; or in copending U.S. application Ser. No. 09/769,964, filed Jan. 24, 2001, and titled “Surgical Instruments for Stabilizing a Localized Portion of a Beating Heart”. U.S. Pat. Nos. 6,036,641 and 6,283,912, and U.S. application Ser. No. 09/769,964 are each hereby incorporated herein, in their entireties, by reference thereto.

At least a second, smaller thoracotomy, stab wound or other opening 106 may be formed in a location removed from primary opening or thoracotomy 102, and is made only large enough to allow support arm 16 to be passed therethrough. The positioning of thoracotomy 106 may be separated far enough from thoracotomy 102 to allow support arm 16 to be inserted obliquely into the working space, as shown, so that support arm does not require much working space for the insertion, i.e. to form a very low profile instrument upon connection with stabilizer foot 12. For example, support arm 16 and stabilizer foot 12 may form an angle ranging from about 90 degrees to about −20 degrees, based on the angle between support arm 16 and the plane of stabilizer foot 12 when contacted to or parallel with the portion of the surface of the heart that is being stabilized.

Stabilizer foot 12 may be provided with multiple connecting members 18 to increase the probability of a connecting member 18 being positioned where support arm 16 can make contact and connect with it, regardless of the orientation of stabilizer 12 after positioning the same. As shown, connecting members 18 each include a ball member 20 that is connected to the stabilizer foot 12 via a stem or connecting element 22 having a reduced cross-sectional area relative to that of ball member 20. In this example, connecting members 18 are rigidly connected to stabilizer foot 12. As shown, connecting members 18 may be oriented so that they extend away from stabilizer foot 12, in orientations approximately aligned with the oblique orientation of support arm 16 as it is inserted through opening/thoracotomy 106, thereby facilitating the connection between support arm 16 and stabilizer foot 12 via a connecting member 18. Connecting members 18 are rigid in the example shown in FIG. 1, but may be made malleable to the extent that they can be reshaped by a user, but requiring a substantial force for reshaping which is greater than any force that will be experienced during stabilization. Thus, even malleable connecting members 18 are rigid during a stabilizing procedure, i.e., are not deformed by stabilization forces or forces applied by the beating heart. Additionally, once it is ascertained which connecting member 18 is to be joined with support arm 16, the other connecting members 18 may be deformed so as to be less obstructive to the surgical site.

FIGS. 12A-12D illustrate stabilizer feet 12 provided with various examples of malleable connecting members 18. In FIG. 12A, at least stem 22 is formed from a malleable material, such as annealed stainless steel or other malleable metal for example, having the characteristics described above, such that the orientation of connecting member 18 may be changed prior to use, but where connecting member 18 has sufficient stiffness so that it will not be deformed during the stabilization process. The example of FIG. 12B shows stabilizer foot 12 provided with multiple connection members 18 attached to a sheet metal layer 12s which is malleable. Connection members 18 may or may not be malleable. If they are not malleable, reconfiguration is still possible by grasping a connection member and applying a bending moment sufficient to plastically deform sheet meal layer 12s in the vicinity where the connection member 18 connects. If the connection members are malleable, then bending as mentioned above may deform both sheet metal 12s and connection member 18. In FIG. 12C multiple connection members 18 are interconnected by wire form 12w which may be of the same malleable material that connection member 18 are made of for example. FIG. 12D shows a variation of the arrangement of FIG. 12B in which sheet metal appendages or extensions 12a extend from sheet metal 12s to provide malleability for re-orienting connection members 18.

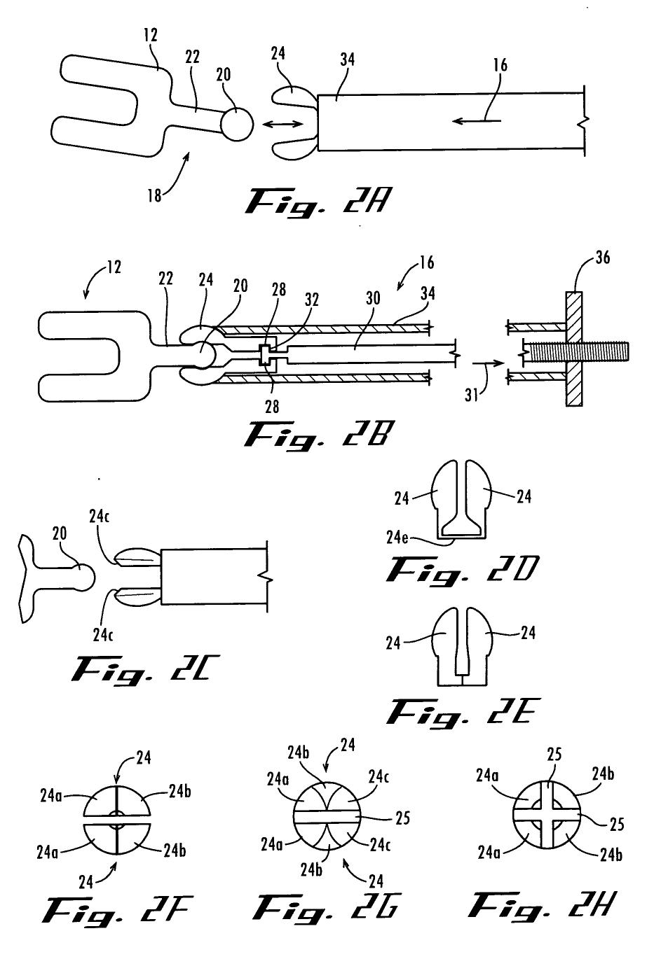

Referring again to FIG. 1, jaws 24 or other grasping/gripping or connecting feature are provided at the end of support arm 16 and adapted to lock onto ball member 20 of any of connecting members 18. FIG. 2A shows a partial view of support arm 16 including jaws 24 and FIG. 2B shows a partial, sectional view illustrating one example of a mechanism for operating jaws 24 to lock to ball member 20. In this example, jaws 24 are made up of a pair of half chucks that open to easily pass over ball member 20. The proximal end portions of jaws 24 each have a shoulder 28 formed therein, and together, shoulders 28 receive anchor 32 formed in the end of tension member 30. An outer tube 34 is provided over tension member 30 and is slidable with respect thereto. In an open position, when outer tube 34 is drawn away, does not apply significant force to or is removed from contact with half chucks 24, half chucks 24 are allowed to spread apart or open to form a distal opening sufficiently large to receive ball member 20. Jaws/half chucks 24 may optionally be spring-loaded or otherwise biased to the open position. Jaws 24 may be further provided with features that assist the lead-in of ball member within the confines of the open jaws 24. FIG. 2C shows one example, where the features in this example are chamfered leading surfaces 24c. Half chucks 24 may be separate pieces, as shown in FIGS. 2B and 2D, or may be joined at their proximal ends 24e as shown in FIG. 2D. The choice between whether to from half chucks 24 as separate pieces or as joined may be based upon manufacturing cost and/or ease of manufacturing. If joined, as in FIG. 2D, then tensioning member may be bolted, welded or otherwise fixed to the proximal end 24e of the joined jaws 24, or may be made integral therewith.

Further, split chucks 24 may be provided, where each half chuck 24 includes two or more split components, such as 24a and 24b in FIG. 2F, or 24a,24b,24c in FIG. 2G. One or more grooves 25 may be maintained between chucks 24, as shown in FIGS. 2G and 2H, when chucks 24 are in the closed position (e.g. when chucks 24 have captured ball member 20) to allow an increased range of angular movement therethrough by stem 22 with respect to the longitudinal axis of support arm 16. Still further, chucks 24 may be mounted to the tensioning member via a rotational joint to allow rotational positioning of the chucks during alignment with and capture of ball member 20. By rotating the chucks, this allows positioning of the slots or grooves 25 between chucks to provide maximum angular positioning of support arm 16 with respect to stabilizer foot 12 in the desired direction. Such a rotational joint may be fixed upon locking jaws/chucks 24 in fixation with the stabilizer foot 12, to maintain stability about the longitudinal axis of support arm 16 for carrying out stabilization.

Upon sliding the distal end of outer tube 34 into jaws 24 and applying force, this causes jaws 24 to move to the closed position (shown in FIG. 2). This action may be precipitated by manually sliding outer tube 34 toward and against jaws 24 while at the same time preventing tension member 30 from traveling in the same direction, or even applying tension to tension member 30 in the direction shown by arrow 31. Alternatively, the proximal end portion of tension member 30 may be threaded, and a mating, threaded drive member 36 may be threaded thereover. Upon advancing drive member (in the direction opposite to arrow 31) along tension member 30 by turning it, this drives outer tube 34 against jaws 24, driving jaws 24 to the closed position shown in FIG. 2. Reverse translation of drive member along tension member 30 (in the direction of arrow 31) releases force on jaws 24 by outer tube 34, thereby returning jaws to the open position so that support arm 16 can be disconnected from stabilizer foot 12.

Further, a light source 26 (FIG. 1) may be provided anywhere in the vicinity of the distal end of support arm 16 to provide light to the user (as viewed through thoracotomy/opening 102 to aid in connecting support arm 16 to stabilizer foot 12, as well as to provide light for performing a surgical procedure, such as an anastomosis with artery 108, for example.

FIG. 3A shows another example of stabilizer foot 12 configured with removable positioning member 14. In this example, tensioning member 42, such as a steel cable, steel fiber or high strength suture, is fixed to foot 12 and threaded through removable positioning member 14. A small enlargement or anchor 44 is fixed on tensioning member 42, against which tensioning forces may be applied to exert tension in tensioning member 42, since anchor 44 cannot pass through slot 44s. An annular space or opening 44a is provided through positioning member 14, and anchor 44 is dimensioned so that it may easily pass through the annulus. After relieving tension in tensioning member 42, anchor 44 may be slid from slot 44s to be passed through space 44a, allowing positioning member 14 to be slid over anchor 44 and removed from thoracotomy/opening 102 after positioning of stabilizer foot 12 has been completed in a manner described hereafter. A large enlargement or stop member 46 is provided at the proximal end of tensioning member 42, which is of a size that cannot pass through the annulus of positioning member 14. Thus, stop member 46 prevents tensioning member 14 from becoming completely separated from the apparatus, as a safety precaution, so that this piece is not misplaced or lost track of. Additionally, if repositioning is desired, stop member 46 maintains positioning member 14 threaded on tensioning member 42, so that positioning member 14 can be readily reattached to stabilizer foot 12.

The process of positioning stabilizer foot 12 includes manually sliding positioning member 14 into contact with stabilizer foot 12 (if it is not already so positioned at the time of inserting stabilizer foot 12 through thoracotomy/opening 102), as shown in FIG. 3B. Anchor 44 is oriented along tensioning member 42 in a position that allows it to be locked against slot 48 on positioning member 14 when positioning member 14 is placed in contact with stabilizer foot 12. By sliding tensioning member 42 through slot 48 so that anchor 44 is on the outside surface of tensioning member 14, slot 48, due to its dimensions, prevents anchor 44 from sliding therethrough. This permits application of tension in tensioning member 42 to lock the position of positioning member 14 relative to stabilizer foot 12. An example of a tension applicator is tensioning nut 50 which may be formed in the proximal portion of tensioning member 14. Mating threads 52 may be provided on the distal portion of tensioning member 14 to mate with threads in tensioning nut 50 (see FIG. 3C). Thus, upon rotation of tensioning nut 50 with respect to the distal portion of positioning member 14 (as indicated by the arrow in FIG. 3B), tension is applied through tensioning member 42 as the length of positioning member increases while anchor 44 maintains a relatively fixed position. With sufficient tension, a rigid connection between position member 14 and stabilizer foot 12 is formed, so that stabilizer foot 12 can be accurately and positively positioned through movements of positioning member 14.

Once stabilizer foot 12 has been positioned where desired, and optionally, a support member 16 has been fixed to stabilizer foot 12 (which will be described below), positioning member 14 may be removed from thoracotomy/opening 102 to increase the working space. Reverse rotation of tensioning nut with respect to the distal portion of positioning member 14 relieves the tension in tensioning member 42, so that tensioning member 42 can be slid back out of slot 48. This allows positing member 14 to be removed from contact with stabilizer foot 12 and slide over anchor 44, removing it from thoracotomy/opening 102.

As shown in FIGS. 3A-3B, the interface between positioning member 14 and stabilizer foot 12 form a ball joint 54, which allows angular positioning/orientation of positioning member 14 relative to stabilizer foot 12, prior to locking the connection. Further, after a certain amount of positioning the user may find it desirable to re-orient the position/angulation of positioning member 14 relative to stabilizer foot 12 to provide an increased or different range of positions that the positioning member 14 is capable of moving stabilizer foot 12 to, given the constraints imposed by the dimensions of thoracotomy/opening 102. This is easily accomplished by relieving sufficient tension, via use of tensioning nut 50, on tensioning member 42 to allow the reorientation of positioning member, after which tension is re-applied to lock the new orientation of positioning member 14 with respect to stabilizer foot 12. As shown, a ball-shaped member 54a is provided on stabilizer foot 12, which receives a cup-shaped member 54b formed in the distal end of positioning member 14. However, these components may be reversed, i.e., a cup-shaped member may be provided on stabilizer foot 12 and a ball-shaped member may be formed at the distal end of positioning member 14.

Alternatively, the assembly may forego the ball joint 54 configuration for use in situations where positioning member 14 may be directly aligned with stabilizer foot 12 through thoracotomy/opening 102 and perform the positioning required without angling the orientation of positioning member 14 with respect to stabilizer foot 12, a joint assembly may be done away with altogether. An example of such an arrangement is illustrated in FIG. 3D, where positioning member 14 is simply locked against stabilizer foot 12 through application of tension by tensioning member 42 as described above. Other alternatives may include other joint interfaces, such as a rotational joint, barrel joint, etc, provided between positioning member 14 and stabilizer foot 12.

Once stabilizer foot 12 has been positioned as desired, support arm 16 may be connected to stabilizer foot 12 via another thoracotomy or other opening 106 and positioning member may optionally be removed from thoracotomy/opening 102 to maximize the working space through thoracotomy/opening 102. If removed, positioning member 14 is preferably not removed from stabilizer foot 12 until a secure connection between support arm 16 and stabilizer foot 12 has been made, such as by fixing jaws 24 to connecting member 18 (e.g., ball member 20) and additionally fixing support arm 16 to a relatively immovable object. Typically, support arm is fixed with respect to the operating table using a standard clamping arm (not shown) that clamps to the surgical table and is adjustable to be moved to the location of support arm 16 wherein it is clamped to support arm 16 and then fixed or locked so that it is relatively immovable. Other alternatives may be employed for fixing the support arm 16 to a relatively immovable object, however, including fixing by hand holding.

Although support arm 16 is shown in FIG. 1 to be a substantially straight and rigid component, alternative support arms 12 may be curved, malleable, or even multi-jointed, to provide a wider range of access to a stabilizer foot 12 to be joined with and/or a wider range of control over which stabilizer foot 12 may be controlled during stabilization.

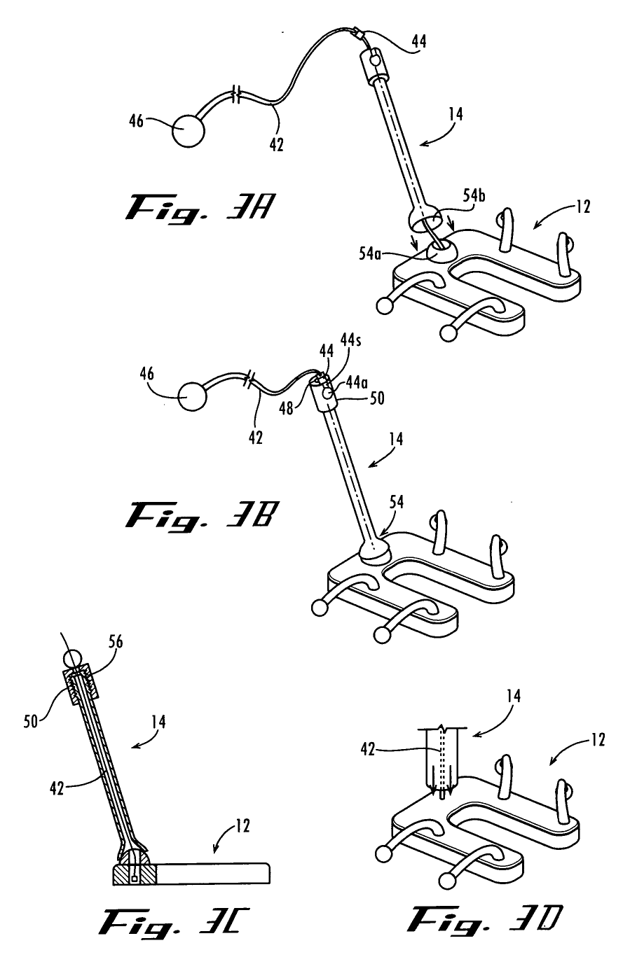

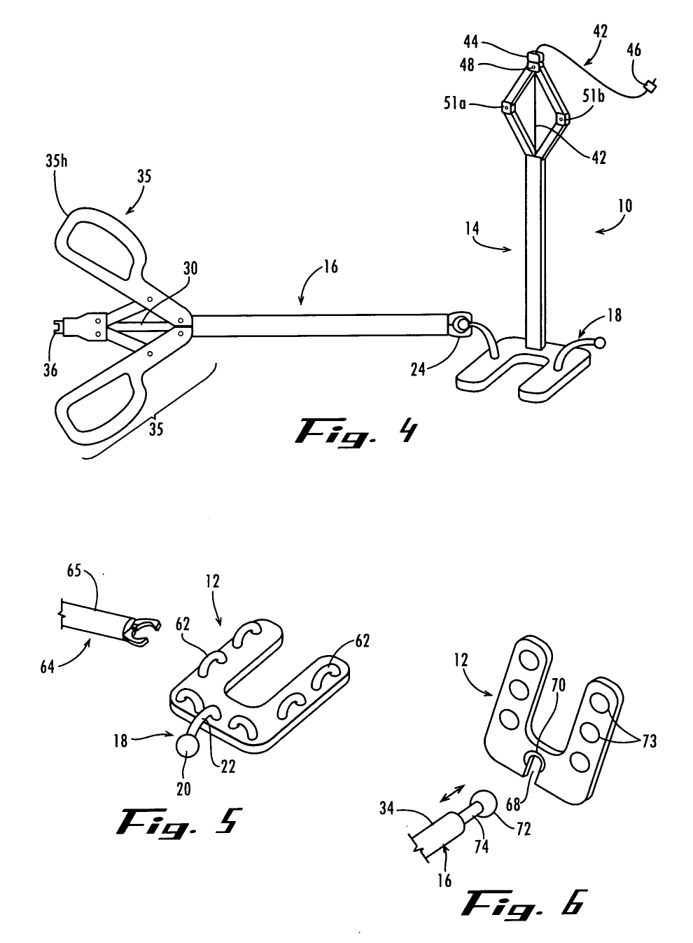

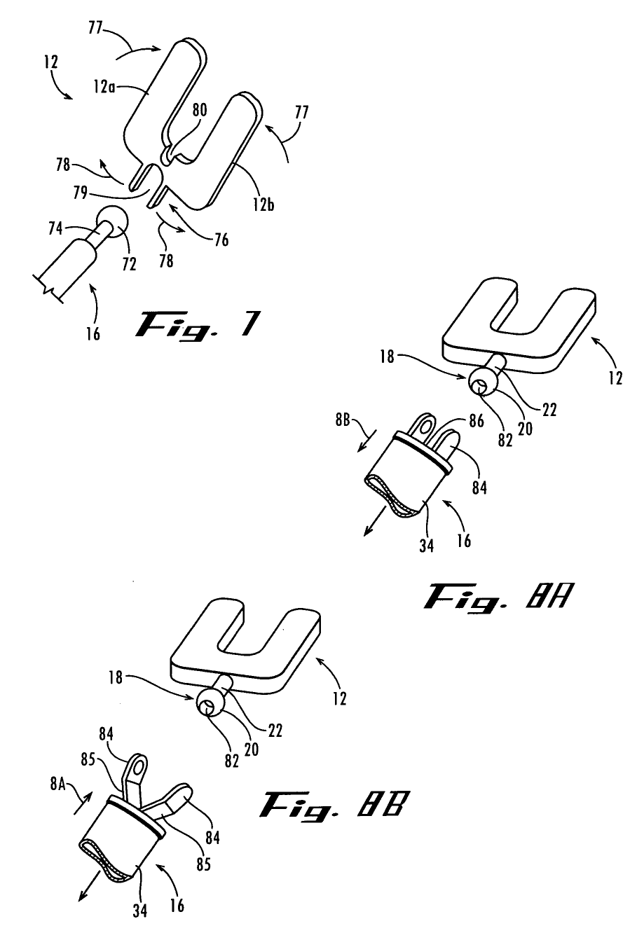

FIG. 4 shows alternative examples of components of a stabilizer assembly 10 which includes alternative locking mechanisms for both support arm 16 and positioning member 14. Also, in this example, stabilizer foot is shown having only two connecting members 18, which may be rigid or malleable, as described above. Jaws 24 may be connected to tensioning member 30 in the same manner as described above with regard to the example of FIG. 2. An adjustment screw or nut 36 is provided at the proximal end of tensioning member 30 which can be rotated to provide fine adjustment of the degree to which jaws close, i.e., the amount of distance left between the jaws may be finely adjusted. Additionally, gross-adjustment mechanism 35 is provided to quickly lock jaws 24 to ball member 20 with a simple action. In the example shown, a pair of handles 35h (like scissor handles) are provided which can be hand-squeezed together by an operator to actuate and lock locking mechanism, which operates in the same manner as the locking mechanism on a VISE-GRIP™ tool. By separating the handles 35h, locking mechanism 35 is quickly released, thereby quickly releasing jaws 24 from ball member 20. Thus locking mechanism is effective in situations where support arm 16 needs to be quickly fixed to stabilizer foot 12, where support arm 16 needs to be quickly released from stabilizer foot 12 and then quickly reaffixed to stabilizer foot 12 (such as if the stabilizer foot 12 needs to be repositioned, for example), or just in general to expedite this part of the procedure. Alternatively, any other “over-center” type locking linkage may be provided for quick locking and releasing of jaws 24 to and from ball member 20.

Positioning member 14 in FIG. 4 is also provided with an over-center type locking mechanism 51 for securely fixing positioning member 14 against stabilizer foot 12. The proximal end of locking mechanism 51 is provided with slot 48 which functions in the same manner as slot 48 in the example described with respect to FIG. 3B. Once tensioning member is positioned in slot 48 to secure anchor 44, linkage components 51a,51b may be squeezed together by hand or with a squeezing tool, such as forceps of the like, effecting a quick locking action of the linkage to place tensioning member 42 under a predetermined amount of tension to lock position member 14 in place against stabilizer foot 12. Although not shown, locking mechanism 51 may also be provided with a fine adjustment mechanism to adjust the amount of final tension that is exerted by locking linkage components 51a,51b. By pulling apart components 51a,51b, this quickly unlocks the assembly, allowing for subsequent removal of tensioning member 42 from slot 48 and withdrawal of positioning member 14 away from stabilizer foot 12 and out of thoracotomy/opening 102.

FIG. 5 shows an example of a stabilizer foot 12 configured for positioning without the use of positioning member 14. In this configuration, a plurality of grab points or grab members 62 are provided around the periphery of stabilizer foot 12 and extend therefrom so that they can be readily grasped by a tool such as grasper 64. Grasper 64 may be a conventional surgical tool and operated by a simple scissor-type actuator, for example, or other conventional, readily available grasping tool dimensioned to be useable through thoracotomy 102. Grab points 62, as shown are partial loops of wire extending from stabilizer foot 12 and fixed thereto at both ends, but may have other configurations, such as tabs extending from stabilizer foot 12, molded configurations that are integral with stabilizer foot 12 but shaped to be easily grasped by grasper 64, other shapes of extensions, or the like. Further, stabilizer foot 12 may be provided with a deformable upper surface, such as by forming it of a viscoelastic material or the like, for example, so that grasper 62 may grasp anywhere on the upper surface of stabilizer foot in order to reposition stabilizer foot 12. A viscoelastic material is advantageous in that after being released by the grasper, the upper surface will relax back to its initial shape.

In use, the stabilizer foot 12 shown in FIG. 5 may be inserted through thoracotomy/opening 102 using grasper 64 and positioned approximately in the desired location on the tissue surface where stabilization is desired. Further adjustment is then carried out by grasping the appropriate grab point 62, moving stabilizer foot 12, releasing the grasp, and grasping another grab point 62, if needed to further reposition stabilizer foot 12. This process can be repeated until the position of stabilizer foot 12 has been finally manipulated into the desired position and orientation.

Further, multiple graspers 64 may be used through multiple thoracotomies to position stabilizer foot using more than one grasper 64, which may provide better and/or faster positioning and control over the positioning of stabilizer foot 12. A third incision, thoracotomy or opening, as well as further additional openings (not shown in FIG. 1) may be provided for access by a second grasper 64 and stabilizer foot 12 may be securely held in position via two graspers through thoracotomy/opening 102 and the third or other additional opening, while support arm 16 is passed through opening/thoracotomy 106 and connected to stabilizer foot 12. When using multiple graspers, one of the grab members may be is grasped by a second grasping tool before releasing a grasp of the same or another grab member by a first grasping tool. Alternatively, grab members may be simultaneously grasped by at least first and second grasping tools to triangulate forces on the stabilizer foot to move the stabilizer foot. Such first and second grasping members may simultaneously engage the same grab member, or, more typically, different grab members.

Due to the large degree of position and orientation control provided by grab points 62, stabilizer foot may only require one connecting member 18, as shown. However, multiple connecting members 18 may be provided, just as in the examples described previously.

FIG. 6 shows a variation in the manner in which support arm 16 may be connected with stabilizer foot 12. It is advantageous to provide a connection which may be easily and rapidly established between support arm 16 and stabilizer foot 12 because closed-chest techniques may necessitate assembly of support arm 16 and stabilizer foot 12 inside the chest, where working space is limited, as has been already described above. In this example, stabilizer foot 12 is provided with a slot 68 with a recess (e.g., a spherical recess or other recess configured large enough to easily receive ball member 72) 70 into which ball member 72 at the distal end of support arm 16 may be inserted to make the connection. Recess 70 functions as a socket forming a ball and socket joint with ball member 72 upon connection of support arm 16 to stabilizer foot 12 as described below. Ball member 72 is contacted to recess 70 on the underside of stabilizer foot 12 as shown. A slot 68 extends from recess 70 to the periphery of stabilizer foot 12 and is wide enough to allow post/shaft 74 to pass therethrough, but narrow enough to prevent ball member 72 from entering or passing therethrough. Upon contacting recess 70 with ball member 72, support arm is then rotated to pass shaft 74 through slot 68 so that support arm is oriented to extend from the top side of support arm 16. After orienting support arm 16 to the desired angle relative to stabilizer foot 12, outer tube 34 is advanced relative to shaft/post 74 and ball member 72, to clamp against the top surface of stabilizer foot 12, thereby locking the relative positions of ball member 72 and recess 70 against one another. Outer tube 34 may be driven using any of the mechanism disclosed herein, see, e.g., the descriptions with regard to FIGS. 2B and 4. Further, outer member is drivable away from stabilizer foot to allow repositioning of support arm 16 relative to stabilizer foot 12, after which the ball joint may be locked again in the manner described above, or, upon further retraction of outer tube 34 away from stabilizer foot 12, support arm 16 may be disconnected from stabilizer foot 12 by reversing the connecting steps described above.

As shown, recess 70 is provided on the bottom or contact surface side of stabilization foot 12. Stabilization foot 12, as shown, is a vacuum- or negative pressure-type stabilizer foot 12, as apparent from the presence of ports 13. However, this same connection arrangement may be applied to other types of stabilizer feet 12, including mechanical-type stabilizer feet 12. Further, although only one slot/recess 68,70 is shown, stabilizer foot 12 may be provided with multiple slots/recesses 68,70 around the periphery thereof, to provide more versatility as to where support arm 16 may be connected.

FIG. 7 shows another variation for connecting support arm 16 with stabilizer foot 12. In this arrangement, stabilizer foot 12 is provided with a socket member 76 extending therefrom. Socket member 76 is preferably bifurcated, but may be configured otherwise as long as it is capable of expanding when the foot members 12a,12b of stabilizer foot 12 are squeezed together. For example, socket member may be segmented into four sections, or trifurcated. In use, stabilizer 12 is inserted through an incision (such as thoracotomy 102) and positioned using any of the techniques described above. Once in the proper position, foot members 12a,12b are then squeezed together in the direction indicated by arrows 77, which causes socket member 76 to expand in the directions 78 shown. In the expanded configuration, ball member 72 can be readily placed within the confines of socket member 76. Upon release of the squeezing forces on foot members 12a,12b, socket member returns to its relaxed, non-expanded state, which is dimensioned to form a secure friction fit with ball member 72. One or more slots 79 are provided by the separations between the socket member components, allowing angulation of support arm 16 with respect to stabilizer foot 12, if necessary, while socket member 76 is in the expanded state. The squeezing of foot members 12a,12b may be performed by forceps or other surgical instrument through thoracotomy 102, for example.

Although not shown in FIG. 7, stabilizer foot 12 may be provided with grab points 62 or other features to facilitate the positioning thereof, and or may be provided with an integral or removable positioning member 14. To assist in the flexing/squeezing of foot members 12a,12b, a cutout or weakened section 80 may be formed between foot members 12a,12b in stabilizer foot 12.

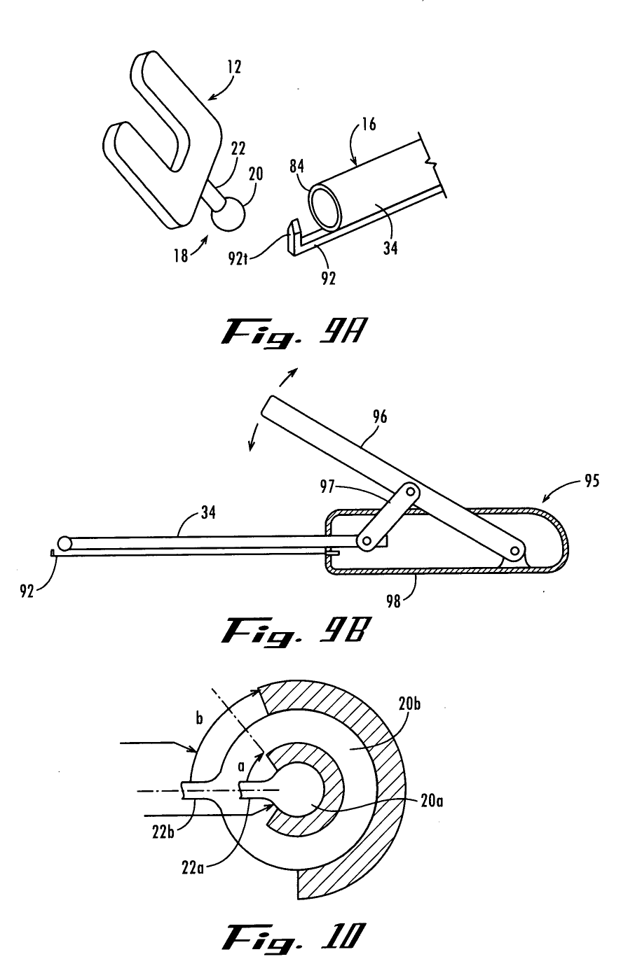

FIG. 8A shows still another variation for connecting support arm 16 with stabilizer foot 12. This arrangement is provided specifically for those stabilizer feet that employ vacuum during the stabilization process. In this arrangement, the need for an external vacuum line is eliminated by providing an airtight support arm through which a vacuum may be applied to stabilizer foot 12. Alternatively, a vacuum line may be run through support arm 16 and connected in an airtight manner to stabilizer foot 12 though the connection arrangement shown. In this arrangement, stabilizer foot 12 includes a connection member 18 having a ball member 20 extending from a stem 22 which connects it with the stabilizer foot 12. In this instance, however, ball member 20 and stem 22 have a central opening or port 82 therethrough which fluidly connect with the features on the contact members of stabilizer foot 12 that are adapted to apply vacuum to the surface of the organ/tissue to be stabilized.

Support arm 16 is provided with a socket member 84 at its distal end, which may be cup-shaped and/or split to form at least one slot 86 to increase the range of angulation with which support arm 16 may be oriented with respect to stabilizer foot 12 and to facilitate spreading of socket member 84 to receive ball member 20 as described below. Preferably, socket member 84 is configured to form a friction fit with ball member 20 upon reducing the inside diameter of socket member 84.

The socket member portions are expandable upon sliding outer tube 34 away from socket member 84 (in the direction of arrow 8B as shown in FIG. 8A to increase the space available for receiving ball member 20, as shown in FIG. 8B. The socket member portions may be biased toward the open position, such as by placement of a biasing member (e.g., coil spring or the like) therebetween (not shown) or may be biased apart as they are engaged against the sides of ball member 20. Further alternatively, a mechanism for positively driving the socket member portions to the open position may be employed, as would be readily apparent to one of ordinary skill in the art. Upon positioning the socket member portions around ball member 20, outer tube 34 may then be advanced in the direction of arrow 8A (see FIG. 8B), wherein the distal end of outer tube 34 rides against ramped or cammed surfaces 85 extending proximally from socket member 84, thereby forcing the portions of socket member toward one another and clamping against ball member 22. Initially, outer tube 34 may be moved distally with respect to socket member only so far as to capture ball member 20 while still allowing relative movement between ball member 20 and socket member 84. This allows the user to position support arm 16 with respect to stabilizer foot 12 into the orientation desired. Further distal movement of outer tube 34 with respect to socket member 84 may then be carried out to apply greater clamping force of socket member 84 on ball member 20 sufficient to lock these members together to prevent any further relative movement.

The ramped proximal extensions may extend further proximally, within outer tube 34 (not shown) and merge into a single rod or shaft for providing driving leverage of outer tube 34 in a manner as shown in FIG. 2B, for example. Other driving mechanisms may be provided for driving outer tube, as discussed herein, and further, as would be readily apparent to those of ordinary skill in the art.

Support arm 16 is further provided with a seal 86 that is mounted at the distal end of outer tube 34 and, together with socket member 84, forms an airtight seal with ball member 20 when socket member 84 is engaged and locked to ball member 20. The connection is made following procedures already described above. Once the connection is made, stabilizer foot 12 is supported by support arm 16 and vacuum may also be applied to the surface against which the contact members make contact by applying a vacuum through support arm 16. This simplifies the process of inserting stabilizer foot through thoracotomy/opening 102, since there is no vacuum line extending from stabilizer foot 12. Further, it eliminates a step which may be carried out with stabilizer feet which do have a vacuum line extending therefrom, where the vacuum line is first inserted through thoracotomy/opening 102 with the stabilizer foot 12 and then manipulated so as to pass out of another thoracotomy, opening or incision to free up space in thoracotomy/opening 102.

As in FIG. 7, and other Figs. in which grab points, a positioning member or other positioning features are not shown on stabilizer foot 12, the stabilizer foot shown in FIG. 8A may be provided with grab points 62 or other features to facilitate the positioning thereof, and or may be provided with an integral or removable positioning member 14. Further, although only one connecting member 18 is provided in the example shown in FIG. 8A, multiple connecting members 18 may be provided extending from different locations on stabilizer foot 12. In this example, when multiple connecting members 18 are provided, each connecting member 18 is provided with a valve in central opening 82 that permits vacuum to be drawn through the opening in a direction exiting stabilizer foot 12 and connecting member 18, but does not allow flow in the opposite direction. In this way, connecting members 18 which are not connected with support arm 16 prevent inflow therethrough when a vacuum is drawn via support arm 16.

FIG. 9A shows a variation of an arrangement at the distal end of support arm 16 for making a connection with a ball member 20 on connecting member 18. In this arrangement, the distal end of outer tube 34 functions as a socket member 84. As shown, socket member 84 is merely an end portion of the cylindrical outer tube 34. Alternatively, the end portion of outer tube 34 may be flared or shaped to more closely resemble a portion of a spherical surface that is dimensioned to form a ball and socket joint with ball member 20. The locking mechanism of this arrangement includes finger 92 that extends distally past the distal end of outer tube 34. Finger 92 is externally mounted with respect to outer tube 34, such as by mounting it in a fixed position relative to driver 95, as shown in the cutaway view of FIG. 9B. Driver 95 includes a locking lever 96 linked 97 to outer tube 34, such that when locking lever 96 is rotated toward handle 98 of driver 95, linkage 97 drives outer tube 34 distally, toward the distal end 92t of finger 92. This is the action that is carried out for locking ball member 20 between finger tip 92t and socket 84. Release of the locking connection is carried out by reverse rotation of locking lever 96 with respect to handle 98. Alternative configurations may be provided to driving the locking mechanism, as will be apparent to those of ordinary skill in the art. Further alternatively, finger 92 may be configured to translate relative to outer tube 34, while maintaining the position of tube 34 stationary relative to driver 95 or other driving mechanism. For example, finger 92 or outer tube 34 may extend beyond the proximal end of outer tube 34 or finger 92, respectively and have threads on the proximally extending portion which mate with a drive nut 36 used to extend and retract finger 92 or outer tube 34, by turning drive nut 36 while drive nut 36 remains in a fixed position along the longitudinal axis of outer tube 34 or finger 92, similar to the concept shown in FIG. 2. Alternative driving arrangements may be used, including ratchet mechanisms, cam drives, and the like, which would be readily apparent to those of ordinary skill in the mechanical arts.

In operation, finger 92 and outer tube 34 facilitate support arm 16 to be quickly and easily connected with and disconnected from stabilizer foot 12. After insertion of stabilizer foot 12 through a first incision and positioning of stabilizer foot on the surface of an organ/tissue to be stabilized, using one or more of the techniques described above, support arm 16, having been inserted through a second incision (either before or after insertion of stabilizer foot 12, described above) is maneuvered to approximate the distal end of support arm with connection member 18. Support arm 16 is maneuvered to a position where ball member 20 resides between distal tip 92t of finger 92 and the distal end of outer tube 34 (i.e., socket member 84), preferably with distal tip contacting ball member 20. Driver 95 is then actuated as described above, to drive socket member 84 into contact with ball member 20 on a surface opposite where distal tip 92t contacts ball member 20, resulting in the ball member 20 being captured between distal tip 92t and socket member 84.

Continued driving by rotation of locking lever 96 may be carried out until a sufficient compression force is developed to lock the relative positions of ball member 20 and socket member 84. As locking lever 96 passes over center of the linkage 97 it locks with respect to driver 95. Lever 96 is pushed against handle 98 and the over-center action locks tube 34 in place against ball 20 and finger 92. Reverse rotation of lever 96 with respect to driver unlocks the mechanism and separates tip 92t and socket member 84 for release of ball member 20. Prior to locking, the user may wish to establish only a slight compression force, where ball member 20 and socket member 84 are in contact, but where the user may still manipulate support arm 16 to angulate its orientation with respect to stabilizer foot 12. Such manipulation may be for purposes of orienting support arm optimally for fixing it to a relatively immovable object, such as the surgical table, for example, or to optimize the orientation of support arm to provide the maximum support to stabilizer foot 12. Once the desired orientation is achieved, support arm is fixed to a relatively immovable object and the compression force is increased to lock the relative positions of ball member 20 and socket member 84.

FIG. 10 illustrates a principle for design of ball and socket type connections that are provided in connections, particularly between support arm 16 and stabilizer foot 12, or between positioning member 14 and stabilizer foot 12. This principle illustrates that the larger a ball member is with respect to the stem on which it is mounted, the smaller the encapsulation angle can be for the same joint strength, therefore, the larger the effective angle of rotation that is available to that stem (as well as a member connected to the stem, such as support arm 16, positioning member 14, or stabilizer foot 12). Thus, for example, the angle of rotation “a” that is available to stem 22a which is attached to relatively smaller ball member 20a is less than the angle of rotation “b” that is available to stem 22b that is attached to the larger ball member 20b. Accordingly, ball member 20 should be made as large as practically possible, relative to stem 22 to provide the greatest range of angulation available for orienting one component relative to another component, in which the components are connected by a ball and socket connection including ball member 20. For example, a typical stem outside diameter used for such purposes is around 0.094″± about 0.010″, and a typical ball member outside diameter is about 0.200″± about 0.015″, although bal member 20 may be made even larger, given that the operating space allows, to take advantage of the above principle where allowed.

Turning now to FIG. 11A, a modified arrangement of a support arm 16 employing jaws 24 for connecting with a stabilizer foot 12 is shown. Jaws 24 are configured to engage with and lock with respect to ball member 20 of stabilizer foot 12, similarly to that described with regard to the jaw members described above. Further, jaws 24 are provided with an additional degree of freedom (such as by the provision of pivot joint 96, for example). Once jaws 24 have made contact with and have captured ball member 20, but prior to locking the connection, support arm may be easily manipulated in both “pitch” and “yaw” axes, thereby improving the maneuverability of arm 16 for optimum orientation, without having to release the grasp of ball member 20, reposition arm member 16 and re-grasp ball member in the new position/orientation of arm 16. Upon locking jaws 24 relative to ball member 20, pivot joint 92 is also locked.

FIG. 11B is a partial view of a mechanism that may be used to operate the pivoted jaws shown in FIG. 11A. The mechanism is shown in an isolated view, with the outer tube of support arm 16 not shown. A pair of control rods 30a,30b are pivotally connected to respective ones of the pair of jaw members 24 at 101a,101b, in addition to the pivot member pivotally connecting the jaw members 24 together, as shown in FIG. 11B. Control rods 30a,30b extend proximally through the outer tube of support arm 16 and may be translated by operator manipulation either distally or proximally with respect to the outer tube. For example, moving rods 30a and 30b both proximally causes jaws 24 to close and moving both rods 30a,30b proximally causes jaws 24 to open. However, by moving rod 30b proximally while moving rod 30 distally, for example, jaws 24 can be manipulated to maintain the same spacing while pivoting, or pitching (upward in FIG. 11B) about pivot 96. Thus, greater control of the jaws 24 is provided, by providing more degrees of freedom about which jaws 24 may be controlled.

FIG. 13A illustrates another arrangement for connecting support arm 16 to stabilizer foot 12 in which socket member 84 is affixed to stabilizer foot 12 and the distal end of support arm 16 includes a connection member 18 comprising a ball member 20 and stem 22. Ball member 20 is expandable, so that when in a contracted, or relatively non-expanded state, it is dimensioned to easily be positioned within the confines of socket member 84, and once so positioned, is expandable to make contact with and then lock its position against socket member 84, thereby fixing the relative positions of stabilizer foot 12 and support arm 16. Socket member 84 preferably includes at least one slot to increase the range of angulation along which stem 22 may be moved therebetween relative to axis L shown in FIG. 13A.

As shown in FIG. 13A, ball member 20 may be formed of a semi-rigid inflatable member such as a polymeric balloon or a thin-walled steel or other metal member that can be hydraulically expanded. Expansion fluid may be delivered through a channel through stem 22 and outer tube 34 or may be delivered through a separate tube that runs externally (not shown) of stem 22 and/or outer tube 34. Alternatively, ball member 20 may be a solid metal or polymer that is a split ball which is expandable by drawing a wedge or cam member 21, as shown in FIG. 13B, to further split ball member 20 and thereby expand its outside diameter. In this arrangement, a cable or other tensioning member 23 is fixed to wedge or cam 21 and tension may be applied through tensioning member 23 (for example, from the proximal end of support arm 26 using a tensioning technique similar to one of those described above) to draw wedge or cam further into ball member 20, thereby expanding it, since ball 20 does not move relative to stem 22 in the direction along the longitudinal axis of support arm 16. Optionally, a biasing member 27 (such as a coil spring or other biasing member) may be placed over tensioning member 23 between the proximal end portion of ball 20 and wedge 21, so that upon release of tension in tensioning member 23, biasing member 27 drives wedge 21 in the opposite direction allowing a reduction of the size of ball member 20 so that ball member 20 can be easily removed from socket member 84.

FIG. 14 is a partial view of a stabilizer assembly 10 which includes a mechanism for magnetically coupling support arm 16 to stabilizer foot 12 or to help guide couplings provided on support arm 16 and stabilizer foot which can then be mechanically locked together, such as by any of the mechanical configurations described herein, or by VELCRO™ or adhesive. By providing powerful permanent magnets, such as rare earth magnets, at the distal end of support arm 16 and a portion of stabilizer foot 12 to connect with the distal portion of support arm 16, these components are automatically drawn together and joined when placed in the vicinity of one another. As shown, magnetic posts 88 are provided at the distal end of support arm 16 and mating recesses such as alignment load bearing holes 90 are provided in stabilizer foot 12 to receive posts 88. Posts 88 are provided with a magnetic polarity opposite that of the material immediately surrounding holes 90, so that post 88 are drawn into holes 90 by magnetic attraction when posts 88 are placed in the vicinity of holes 90. Thus, magnetic posts 88 key into holes 90 in stabilizer foot 12 thereby providing mechanical support by the connecting mechanism in addition to the magnetic support. Although stabilizer foot 12 is a suction type stabilizer foot as shown in FIG. 14 with a suction tube 91 supplying negative pressure to ports 13, it is noted that the magnetic/mechanical connection mechanism described may be similarly applied to other types of stabilizer feet including mechanical type stabilizer feet.

Support arm 16 as shown in FIG. 14 is a multi-link arm formed of multiple articulating links which render it flexible in a relatively untensioned state. A cable 23 and tensioning mechanism (not shown) are provided for applying tension to the links to lock support arm in an assumed configuration. The support arm 16 in the embodiment of FIG. 14, as well as in other embodiments described herein, may take alternate forms, such as a straight rigid shaft, a curved rigid shaft, a curved rigid tubular member, and adjustable length shaft or tube, a malleable elongated member, or other arrangements of multiple links including multiple ball joint members, multiple links, multiple alternating links and balls.

Examples of alternative support arm configurations that may be used and/or modified for use with the presently disclosed devices can be found, for example, in U.S. Pat. Nos. 6,290,644; 6,315,717; 6,394,951 and 6,673,013, as well as in copending U.S. application Ser. No. 09/769,964. U.S. Pat. Nos. 6,290,644; 6,315,717; 6,394,951 and 6,673,013 are each hereby incorporated herein, in their entireties, by reference thereto, and U.S. application Ser. No. 09/769,964 has already been incorporated by reference above.

Whatever the form of support arm 16, it is preferably, although not necessarily connected to the connecting base 89 for posts 88 by an articulating joint such as a ball and socket arrangement 93. Such a connection provides more flexibility for alignment of posts 88 with holes 90. However, as noted, an articulating joint is not absolutely required, as support arm may be directly fixed or integrally joined with base 89.

FIG. 15 shows an example of a stabilizer assembly in which one or more vacuum lines 91 that are used to supply negative pressure to a vacuum-type stabilizer foot 12 are also used to guide and align the connecting mechanism for joining support arm 16 with stabilizer foot 12. A yoke 94 is fixed with respect to a distal end portion of support arm 16 and is configured to slide over one or more vacuum lines 91 thereby maintaining jaws 24 in alignment with ball member 22 with which the connection will be made. In the example shown, yoke 94 is threaded over two vacuum lines 91 which provide very accurate alignment of jaws 24 with ball 22. However, the same principle may be applied where only one vacuum line 91 is employed, whereby yoke 94 would slide over the single vacuum line to assist in maintaining the alignment of the connecting mechanism. Also, although jaws 24 and ball member 22 with stem 20 are shown as the connecting mechanism in FIG. 15, other connecting mechanisms as described herein may be substituted, while still applying the alignment mechanism discussed with regard to FIG. 15. Further, although a multi-link support arm is shown in FIG. 15, other alternative support arm configurations may be substituted, as described above. Still further, this same concept may be applied by providing a wire or tube other than a vacuum line over which support arm 16 may be guided to direct alignment of the connecting elements between support arm and stabilizer 12. This is particularly applicable to mechanical stabilizer assemblies in which no vacuum line is used, but may also be used in vacuum stabilizer assemblies, in addition to, or alternatively to use of one or more vacuum lines as a guide.

In use, stabilizer foot 12 and vacuum lines 91 may be inserted through primary opening/thoracotomy 102 and then the proximal ends of surgical tubes are drawn into the surgical site and then routed back out through a secondary opening, such as opening 106 or another opening, thereby removing vacuum lines 91 from the primary opening 102 and establishing them through the opening that support arm 16 is to be inserted through. Next yoke 94 is threaded over vacuum lines 91 and vacuum lines 91 are connected with a source of vacuum. After at least grossly positioning stabilizer foot in a desired orientation, support arm is advanced toward stabilizer foot, guided by the interaction of yoke 91 with vacuum lines 91, until jaws 24 are positioned to lock over ball member 20. Upon locking the connection, any further positioning of the stabilizer foot is accomplished if needed, and then a vacuum is applied via vacuum lines 91 to fix stabilizer foot 12 to the surface or the organ. Support arm is then locked to a relatively stationary object, as described above. When a flexible support arm 16, such as a multi-link support arm is employed, the arm itself may be locked prior to connecting jaws 24 to ball member 22, prior to further positioning after connecting jaws to ball member 22, or just prior to fixing the support arm 16 to a relatively stationary object, depending upon the circumstances of the particular procedure being performed.

FIG. 16 shows a stabilizer assembly 10 that does not require support arm 16 in order to stabilize a portion of the organ (such as a beating heart) to which it is attached. Stabilizer assembly 10 of FIG. 16 employs a vacuum-type stabilizer foot 12 capable of attaching to the organ through the application of vacuum, as described previously. One or more vacuum tubes 91 are fluidly connected with stabilizer foot 12 to lead out of the patient's body and connect with a source of vacuum to deliver negative pressure to stabilizer foot 12 for establishing suction between stabilizer foot 12 and the surface of an organ to be stabilized. Vacuum tube(s) 91 may extend out of the primary opening 102 through which the stabilizer foot has been inserted, or may be rerouted through another opening to provide maximum working space through primary opening 102.

Soft tissue retracting tapes 97 are attached or fixed to stabilizer foot 12 and extend therefrom, as shown. In use, stabilizer foot 12 is passed through opening 102 and maneuvered to the desired position and orientation on the surface of the organ in a location where stabilization is desired. Although not shown in FIG. 16, any of the configurations for grab members 62 or connecting members 62 may be provided on stabilizer foot 12 to assist in properly positioning stabilizer foot in the desired location and orientation. Alternatively or additionally, the superior surface of stabilizer foot 12 may include a viscoelastic layer as described above. Once properly positioned and oriented, vacuum is applied to stabilizer foot 12 via vacuum line 91, whereby the stabilizer foot fixes to the surface of the organ in the desired location, and any graspers 64 employed for positioning stabilizer foot 12 are removed from any openings that they had been inserted through to access stabilizer foot 12.

Tension is next applied through soft tissue retracting tapes 97 to draw stabilizer foot 12 and the attached surface of the organ toward opening 102 by a distance that substantially stabilizes the surface of the organ, particularly between the members 12a and 12b of stabilizer foot 12. Soft tissue retracting tapes are then fixed relative to the patient, by fixation outside of the opening 102. For example, soft tissue retracting tapes may be provided with an adhesive 97a on the side adjacent the patient's skin so that soft tissue retracting tapes can be adhered to the skin of the patient to maintain the tension on stabilizer foot 12. Other means of fixation may be substituted, such as sutures or a retraction mechanism external to the patient, for example. Thus, localized stabilization is accomplished without the need to fix stabilizer foot 12 to a support arm 16 that is in turn fixed to a relatively stationary object.

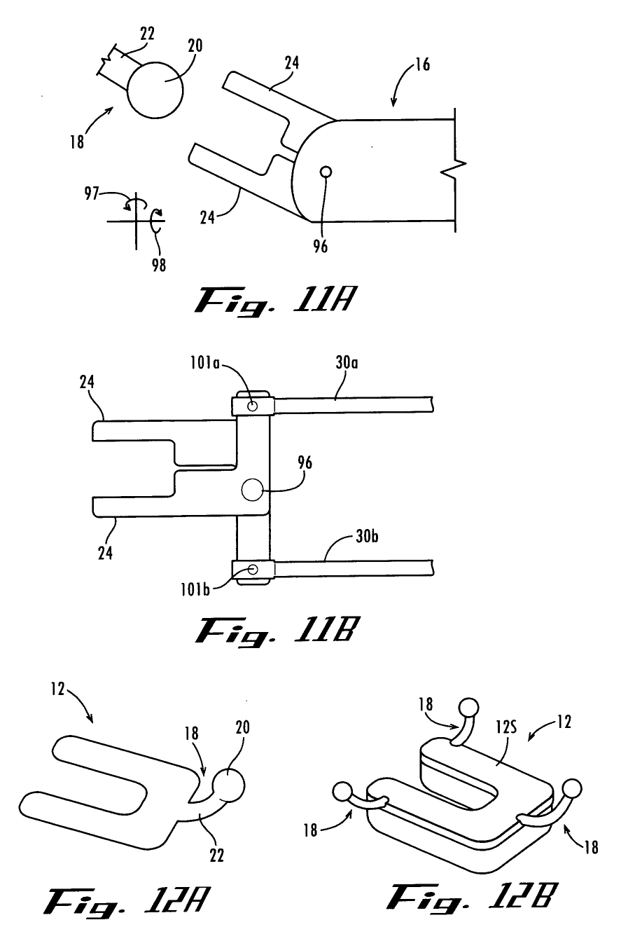

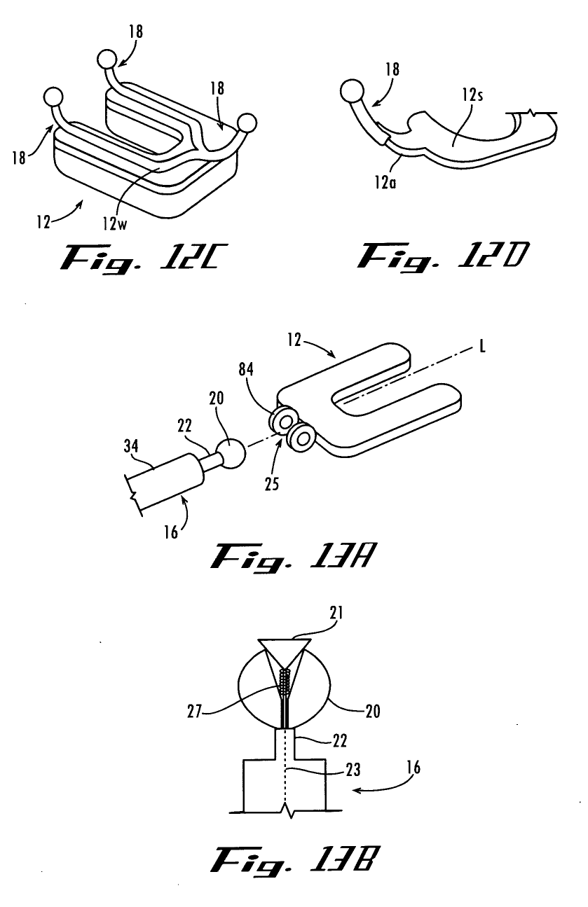

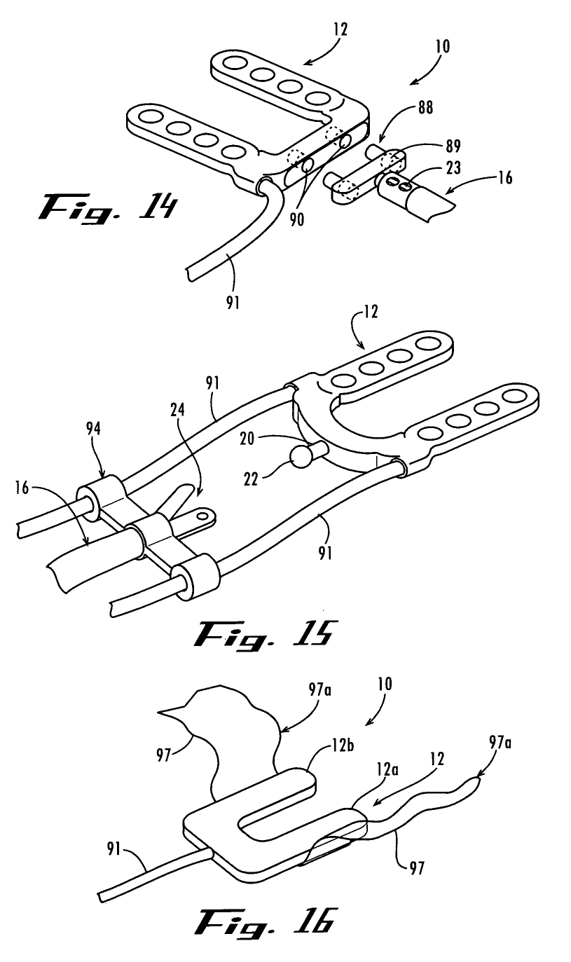

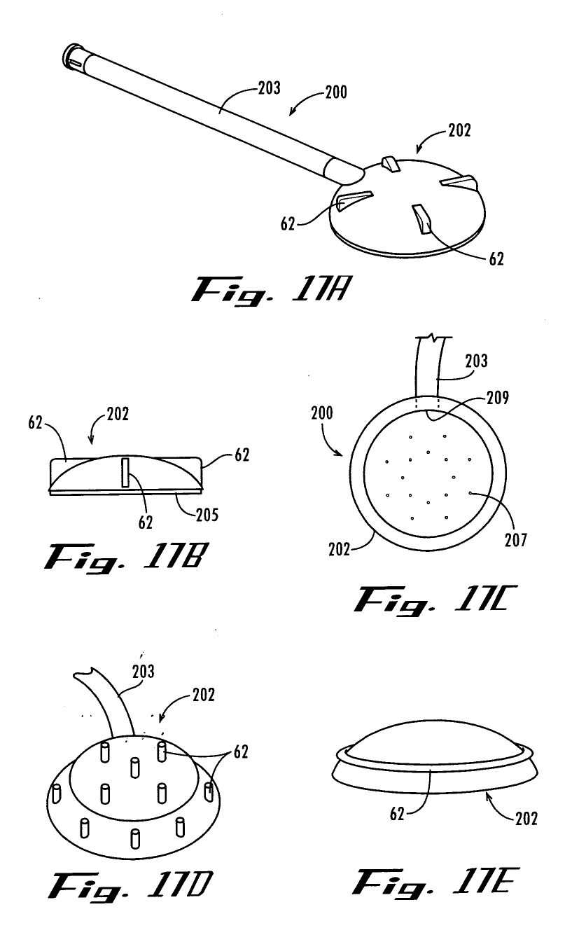

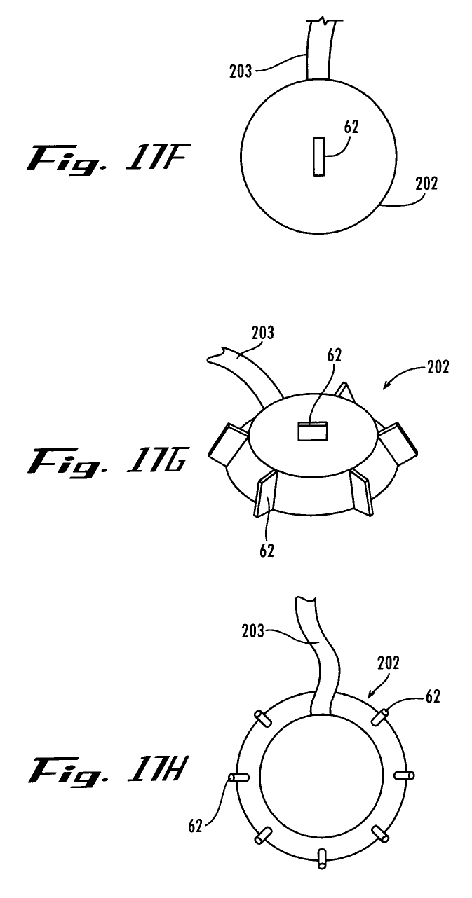

Turning now to FIG. 17A, an organ manipulator 200 for use in closed-chest or limited space surgical sites is shown. Manipulator 200 includes a low profile suction member 202 configured to engage with and maintain a grasp of an organ, such as a heart for example, with a sufficient strength so as not to break contact when suction member 202 is moved, so that the organ is moved by moving suction member 202. As shown, suction member 202 is a low profile suction cup, which may be made of silicone or other biocompatible elastomer sufficient to maintain the suction between suction member 202 and the organ as the organ is moved via movement of suction member 202. Further alternatively, suction member 202 may be made up of more than one suction cup, or other multiple members adapted to apply negative pressure, such as multiple arms having ports, for example.

Suction member 202 may be further provided with a plurality of grab members 62 around the periphery or spaced about the superior surface thereof, that extend from the superior surface of suction member 202 so that they can be readily grasped by a tool such as grasper 64. A typical grasper 64 includes a small diameter shaft 65 (e.g. in the vicinity of 5 mm outside diameter) which extends the jaws of the grasper through an opening 102, 106 or other opening, while permitting an operator to operate the jaws from outside the patient to grab and release grab members 62 in the process of effectively moving and/or repositioning the instrument from which grab members 62 extend. Grasper 64 may be a conventional surgical tool and operated by a simple scissor-type actuator, for example, or other conventional, readily available grasping tool dimensioned to be useable through thoracotomy 102 or other opening or port through the chest wall. As shown, grab members 62 are configured as semi-circular flaps or fins that are integrally molded with suction member 202 to extend therefrom. However, grab members 62 may have other configurations, such as loops or other extensions extending from suction member 202 that may be easily grasped by grasper 64. Grab members 62 are typically grasped to position organ manipulator 200 on an organ in a desired position and orientation prior to applying suction, buy may also be used to move organ manipulator 200 and the organ after suction has been applied and organ manipulator 200 has become fixed to the organ. When using multiple graspers, one of the grab members may be is grasped by a second grasping tool before releasing a grasp of the same or another grab member by a first grasping tool. Alternatively, grab members may be simultaneously grasped by at least first and second grasping tools to triangulate forces on the suction member foot to move the suction member during positioning or manipulation. Such first and second grasping members may simultaneously engage the same grab member, or, more typically, different grab members.

Further, each suction member 202 may be provided with seal 205, as shown in FIG. 17B, which may be made of closed-cell foam that enhances the ability of suction member 202 to conform to the surface of the organ and to establish a vacuum seal therewith. FIG. 17B also illustrates a side view of one configuration of grab member 62. Suction member may also be provided with a layer of open-cell foam 207 or mesh, as shown in FIG. 17C, to act as a diffuser and prevent or substantially reduce the possibility of the surface of the organ from being sucked against one or more ports 209 that establish the vacuum seal. Although shown with only one port 209 entering from the periphery of suction member 202, suction member 202 may alternately be provided with a port that enters suction member from the center or top of suction member 202. Further, suction member 202 may be provided with a plurality of suction ports 209 to deliver negative pressure to the interior thereof. Further design variations that may be applied to suction member 202 are described in co-pending, commonly assigned application Ser. No. 10,615,007 filed Jul. 8, 2003 and titled “Organ Manipulator Apparatus”, in U.S. Patent Application Publication No. 2003/0009080 A1, and in U.S. Pat. No. 6,338,712. Application Ser. No. 10,615,007; U.S. Patent Application Publication No. 2003/0009080 A1; and U.S. Pat. No. 6,338,712 are each hereby incorporated herein, in their entireties, by reference thereto.

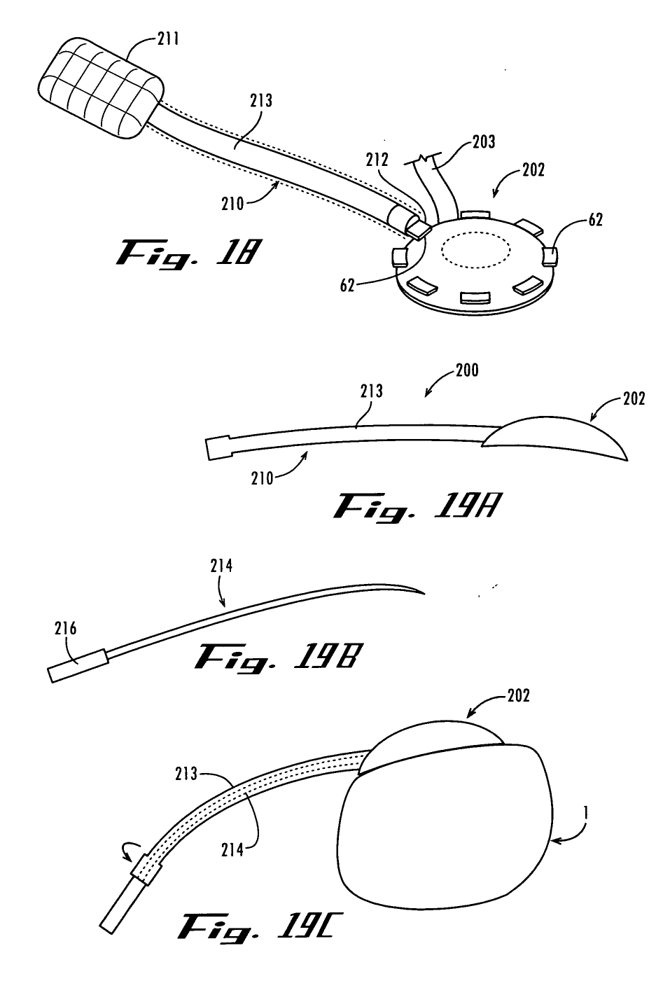

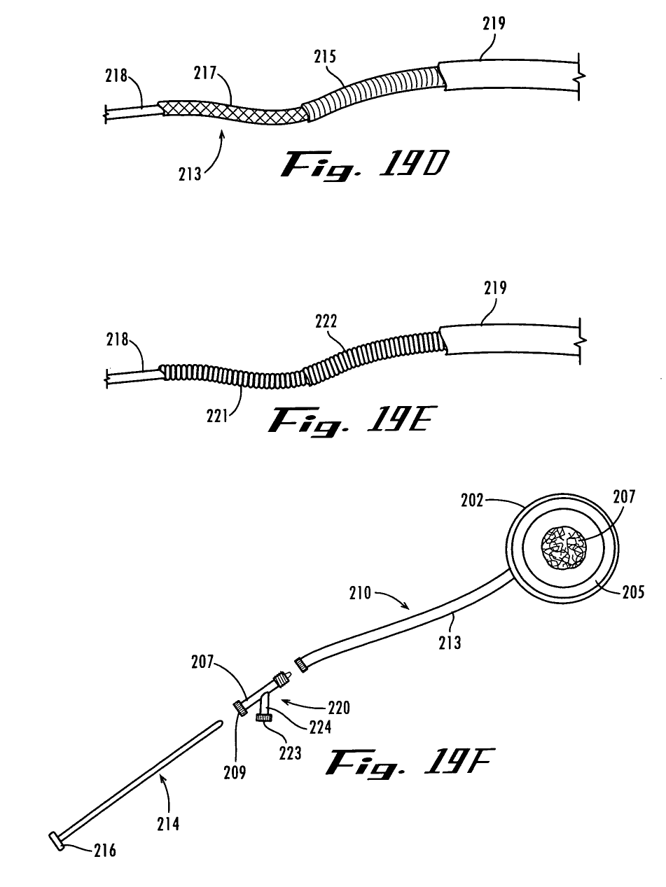

Manipulator 200 may further include an elongated member 203 including a vacuum line 203 extending from suction member 202 for delivery of negative pressure to suction member 202 from a location outside of the body. Elongated member 203 may be a flexible, air impermeable tubing or alternatively may further employ a structure for facilitating manipulation of suction member 202, such as a torque tube which is flexible in bending, but resistant to torquing motions. Still further, a stylet or other shaping member may be inserted through member 203 to maintain elongated member in a desired shape. Further details about torque tubes/positioners and shaping members suitable for use here are described below with regard to FIGS. 18-19F.

In one embodiment, manipulator 200 includes a flexible suction member 202 with a substantially circular opening having an inside diameter of about one to about one and a half inches and an outside diameter of about one and a half to about two inches, with seal 205 extending from the bottom surface of the flexible member 202 by a thickness of about ⅛ to ⅜ inches. Elongated member 203 extends from suction member 202 by about twelve to eighteen inches, and comprises a torque tube that is flexible in bending but resistant to torquing motions about the longitudinal axis of elongated member 203. It should be noted here that these dimensions are with regard to a particular embodiment, and may vary depending upon the patient, size of the organ to be manipulated, and location of entry though which the manipulator 200 is inserted, etc.

FIG. 17D shows a manipulator assembly 200 in which grab members 62 are formed as extending nubs and extend from locations distributed over the superior surface of suction member 202, i.e., are not limited to distribution around the perimeter of the upper surface. FIG. 17E shows another variation of grab members 62 in which a continuous, ring-like projection 62 is formed around the suction member 202 to extend therefrom. FIG. 17F shows still another variation in which a single tab-like grab member extends from the center or top of the superior surface of suction member 202. The example in FIG. 17G includes suction member 202 having a tab or flap on the superior surface thereof, as well as rectangular shaped flaps 62 extending radially from the periphery of suction member 202. FIG. 17H shows another arrangement in which nubs 62 extend radially from the periphery of suction member 202.

Referring now to FIG. 18, manipulator 200 includes grab members 62, such as flaps, nubs or the like, as described above. FIG. 18 shows use of positioner 210 to grab or grasp a grab member 62 and then translate and or rotate positioner 210 to move and reposition suction member 202. After moving with a first grab member 62, another grab member 62 may be grabbed by positioning member 210 and moved as described to extend the range over which positioner 210 may move suction member, without requiring more space for moving the shaft 213 of positioner than is provided by the opening through which positioner 210 has been inserted. This process may be repeated with multiple grasp members 62 until suction member 202 has been moved to a desired location and orientation. Once the desired orientation has been achieved, vacuum may be applied though vacuum line 203 to fix the position and orientation of suction member against the surface of the tissue or organ upon which it has been placed and oriented.

Positioner 210 may include handle 211 at the proximal end that is hand controllable, outside of the patient, to control distal end 212 (jaws) for positioning movements against grasped grab members 62. The shaft 213 of positioner 210 may be malleable so that it can be reshaped as desired to improve the range over which distal end 212 is capable of contacting grab points 62. Reshaping of malleable shaft 213 may be performed between positioning movements for example, by withdrawing positioner 210 from an opening through which it has been placed to accomplish a first positioning movement, reshaping malleable shaft 213 by bending it into a desirable configuration, and reinserting positioner 210 through the same or a different opening to perform another positioning movement.

In use, suction member 202 may be folded into a cylindrical shape and passed through an opening (e.g., opening 102) to be placed on the surface of an organ to be manipulated. Vacuum line 203 may be pulled into the operating space and rerouted though another opening to remove it from obstructing the working space in the opening through which suction member was passed, and/or to provide a desired orientation/direction of vacuum line 203 for use in effecting the movement of the organ, described below. Alternatively, vacuum line 203 may be maintained in its position extending through opening 102 to deliver vacuum to suction member Suction member is then maneuvered to a desired position and orientation on the surface of the organ in a location judged best for applying leverage to the organ to move the organ to a desired orientation/location. As described previously, grab members 62 may be grabbed and pulled, pushed and/rotated to effect the desired positioning of suction member 202.

Once suction member has been properly positioned and oriented, vacuum is applied to suction member 202 via vacuum line 203, whereby suction member 202 fixes to the surface of the organ in the desired location with sufficient strength so that suction member 202 can be moved to move the organ without losing its grasp on the organ. Any graspers 64 employed are removed from any openings that they had been inserted through to access suction member 12. As noted above, shaft 113 of positioner 201 may be malleable so that it can be reshaped as desired to improve the range over which suction member 202 may be placed on the heart.

Tension is next applied on suction line 203 in sufficient amounts to pull and move suction member 202, thereby also moving the organ to the desired position or location or orientation. Suction line may then be fixed to a relatively stationary object outside of the patient, or may be hand held to maintain the desired orientation of the organ until such orientation is no longer desired, such as when a surgical procedure has been completed. Suction member 202 may be released and repositioned on the organ if a further procedure is to be performed where the organ needs to be placed in a different location or orientation.

FIG. 19A is another example of manipulator 200 having positioner 210 which is connected to suction member 202 and which has a shaft 213 that is very flexible in bending but very stiff with regard to torsion. This is made possible by overlaying a coil 215 (preferably a metallic coil, e.g., stainless steel or the like) over a braided tube 217 (see partially cutaway view of FIG. 19D) which is also preferably made of metal, such as stainless steel or the like. Tubular braid 217 is formed over an inner polymeric sleeve 218, which is preferably made of a polymer. Braid 217 is typically encapsulated in sleeve 218, such as by molding, extrusion, forming, or the like. Sleeve 218 may be made from PEBAX® resins (polyether-block co-polyamide polymers), nylon, silicone, urethane or other flexible, biocompatible polymer, or combinations thereof, for example.

An outer polymeric sleeve 219 may be provided over coil 215, as also shown in FIG. 19D. Similarly, coil 215 is encapsulated in sleeve 219, such as by molding, forming, extrusion or the like. Sleeve 219 may be made from any of the polymers described with regard to sleeve 218. The polymeric sleeves function to maintain vacuum integrity, i.e., so that no significant leakage of vacuum occurs along the sleeves 218,219. Additionally, by embedding the coil and braid components into the respective sleeves, the sleeves add significant mechanical support to the coil and braid. This configuration provides a tubular structure that is very flexible with regarding to bending (e.g., with respect to it's longitudinal axis), but very stiff in torsion, so that torquing of tubular member by an operator will transfer the torsional forces effectively to suction member 202 to effect repositioning. Alternatively, braid 217 may be slid over sleeve 218 and/or sleeve 219 may be slid over coil 215 or heat shrunk thereon, but if the coil and braid are not embedded in the sleeves, then the mechanical support by the sleeves decreases substantially.

FIG. 19E shows another configuration for positioner shaft 213 that is also very flexible in bending but stiff under torsion. In this example, oppositely wound spring coils 221,222 (preferably metallic, such as stainless steel of the like) are provided in the shaft. For example, a counterclockwise inner spring 221 may be provided over inner polymeric sleeve 218, and a clockwise outer spring 222 may be provided over inner spring 221. Like the previous example, an outer sleeve 219 is provided over outer spring 222. Of course, this arrangement may be altered so that inner spring 221 is clockwise wound and outer spring 222 is counterclockwise wound. Spring 221 is typically encapsulated into sleeve 218 and spring 222 is typically encapsulated into sleeve 219, such as by any of the techniques described above with regard to the previous example. However the springs and coils may alternatively be slid into place over one another, although some mechanical support by the sleeves in such a configuration may be diminished.

The aforementioned configurations are particularly useful for endoscopically delivering and positioning the manipulator device 200 and subsequent attachment to an organ and manipulation thereof. Placement of the manipulator and maneuvering of suction member 202 are improved with the use of a rigid and/or malleable stylet, an example of which is described hereafter.

A malleable stylet 214 is shown in FIG. 19B that is configured to be passed within the tubular shaft 213 of positioner 210. Stylet 214 may be made from a malleable metal, such as such as annealed stainless steel, nickel-titanium alloy or malleable polymer, for example. Stylet 214 can be bent into any desirable curvature or compound curvature desired, and retains sufficient stiffness after bending to force tubular shaft into a conforming curvature when stylet 214 is passed therethrough. Stylet 214 may be provided with a handle 216 at its proximal end to facilitate use and manipulation thereof for insertion through tubular shaft 213, as described below.

By bending the stylet 214 as desired and inserting it into tubular shaft 213, an optimal or near optimal orientation angle of approach of the inferior surface of suction member 202 can be established with respect to the location of the organ to be grasped, as illustrated in FIG. 19C. Further, once the organ 1 has been contacted and fixed by suction member 202, as shown in FIG. 19C, rotation of torsionally stiff tubular shaft 213 transfers the rotational force to suction member 202 to turn it and the organ with it.

FIG. 19F shows an exploded view of manipulator 200, stylet 214 and a valved connector 220 used to facilitate insertion of stylet through tubular shaft 213, as well as to fluidly connect manipulator 200 with a source of vacuum. Connector 220 may be connected to positioner 210 via a Luer connector or other well-known connection fitting that provides a vacuum-tight seal between the components. Suction member 202 may be provide with one or more ports (not shown) which may be fluidly connected, via tubular shaft 213 and vacuum connector 224/vacuum port 223. Vacuum connector 224 is provided as an integral branch of connector 220.

A second branch 207 of connector 220 provides a port 209 for inserting stylet 214 therethrough in a manner as described above. Port 209 includes a seal therein for making a vacuum tight seal with the shaft of stylet 214 upon insertion therein. Additionally, branch 207 is provided with a valve, so that stylet may be removed from port 209 and the manipulator may continuously provide vacuum to suction member without loss of vacuum pressure through port 209. With this configuration, vacuum may be applied to suction member 202 by connecting vacuum port 223 with a source of suction, and vacuum may be maintained simultaneously with the use of (including insertion and/withdrawal of) stylet 214. This provides the user with a great deal of flexibility, as it may be preferred, in some instances to remove stylet 214 after positioning suction member 202 and establishing a grasp of the organ in the desired place, where afterwards, further maintenance of positioning and/or manipulation may be accomplished by applying tension through positioner 213 without the use of stylet 214. On the other hand, in some instances it may be desirable to leave stylet 214 in the inserted position, or to reinsert it after a removal, to aid in further manipulation. All of these options are available, as facilitated by the arrangement of connector 220.

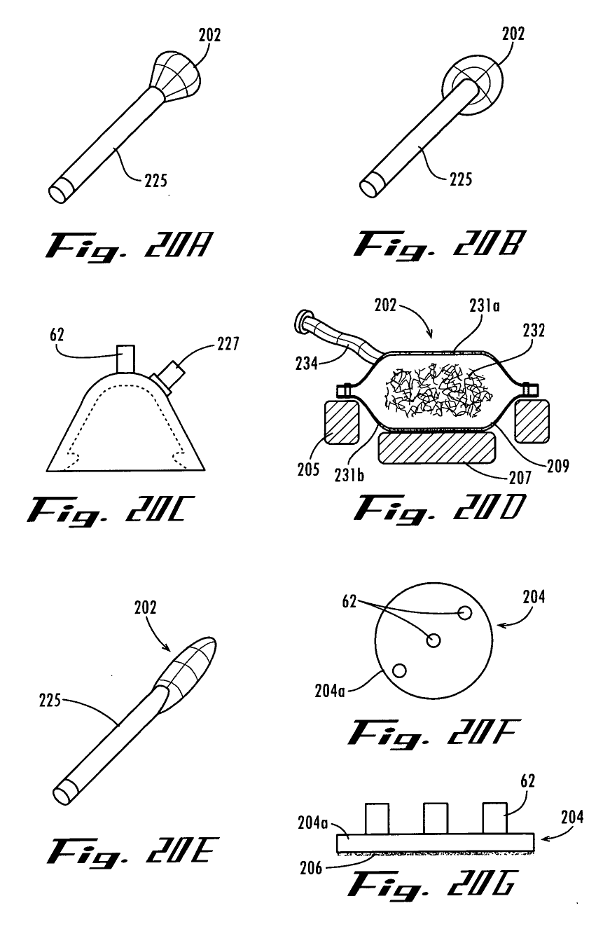

As already noted above, manipulation of an organ, such as the beating heart, other organ or stopped heart, while operating through an opening smaller than a full sternotomy generally requires a manipulator with size and profile optimized for the surgical space that is available in which to perform manipulation. FIGS. 20A-F show various of examples of suction members that may be employed in a manipulator to perform manipulation in a limited surgical space. These various configurations offer some different advantages, and may be chosen for use based upon the available space for performing the manipulation.

FIGS. 20A-B show examples of relatively high profile suction members 202 (e.g., suction cups) that are fixed to rigid positioning shafts 225. Vacuum may be provided to the suction members 202 of each of these examples through the positioning shafts 225 by connecting the proximal end of the positioning shaft (eternal of the opening) to a source of vacuum. The manipulator of FIG. 20B is configured so that suction cup 202 is axially aligned with shaft 225, while the example of FIG. 20B provides shaft 225 mounted to the apex of suction cup 202 at approximately a right angle. Choice of use between these two configurations depends upon the angle of access that an opening provides, with respect to the surface of the tissue/organ to be grasped by suction member 202 for performing manipulation. In either case, a fairly generous amount of space is required above the surface to be grasped in order to employ these relatively high-profile suction members. However, when it is possible to employ one of these configurations, rigid, positively attached positioning shaft 225 provides excellent positive positioning control of movement of suction member 202 (and grasped organ/tissue) both with pushing and pulling motions, as well as rotations, twisting and other angular movements within the limits set by the opening through which positioning shaft 225 extends.

FIG. 20C shows a sectional view of a suction member 202 that is similar to the suction members of FIGS. 20A-20B, in that it is a relatively high profile suction cup, but this example lacks the rigid positioning shaft of the examples of FIGS. 20A-20B. Rather, at least one grab member 62 is provided to extend from the external surface of suction member 202 to be grasped and manipulated in the manner described above with regard to FIGS. 17A-18. Use of this configuration may be selected where there is sufficient surgical space vertically above the organ/tissue to be manipulated, but where the opening to be worked through, and/or peripheral space around the organ/tissue to be manipulated, limits the amount of lateral and/or rotational movement that would be needed to effectively manipulate using a rigid positioner 225. In using the device of FIG. 20C, suction member is first inserted through an opening and grossly positioned on the organ/tissue to be manipulated. Next, positioning and orientation of suction member into the desired placement is performed using one or more graspers, for example as described above. Once properly positioned and oriented, negative pressure is generated between suction member 202 and the organ/tissue, by Inserting a syringe through valve 227, drawing a vacuum, and then removing the syringe. Manipulation may then be accomplished by moving suction member 202 through additional grasping and manipulation with a grasper.

A sectional view of a low profile suction member 202 is shown in FIG. 20D. Rather than a conical or cup-shaped configuration, suction member 202 in FIG. 20D is provided with a more flattened configuration. A pillow-shaped main body portion 230 includes superior and inferior flexible membranes 231a,231b separated by a dispersing material 232, such as felt or other resilient porous material to ensure maintenance of vacuum flow through main body 230 and prevent collapse of the membranes 231a,231b against one another when a vacuum is drawn thorough suction conduit 234. Membranes 231a,231b may be made from polyurethane film or thin laye4rs of any flexible, biocompatible polymer wherein films of the polymer can be joined by direct application of heat, radiofrequency energy, ultrasound energy, microwave energy or laser, for example. Seal 205 may be provided around the perimeter of inferior flexible membrane 231b, and may be made of closed-cell foam, for example, to enhance the ability of suction member 202 to conform to the surface of the organ and to establish a vacuum seal therewith. Suction member 202 may also be provided with a layer of open-cell foam 207 or mesh within the confines of seal 205, to act as a diffuser and prevent or substantially reduce the possibility of the surface of the organ from being sucked against one or more ports 209 provided through inferior membrane 231b that deliver the vacuum to the surface of the organ to establish the vacuum seal.

Vacuum conduit 234 may be configured in a flexible but torsionally rigid shaft 213, such as described above with regard to FIGS. 19A and 19C-19E, or may be simply a flexible vacuum tube. However, when only a flexible vacuum tube is employed, suction member 202 may need to be provided with features to facilitate placement and orientation, e.g., grab members 62, and manipulation through use of a simple vacuum tube is limited to application of tension only.

FIG. 20E is a perspective view of another low profile manipulator in which suction member may be constructed similarly to that described above with regard to FIG. 20D. In the example shown, suction member 202 is oval-shaped, although circular or other shaped suction members may be employed in this type of configuration as well. In this example, a rigid positioning shaft 225 is fixed to suction member 202 and also functions to deliver the vacuum thereto. Accordingly, when the surgical space is somewhat limited, but the opening through which the manipulator is to be inserted allows a fairly straight line approach to positioning, orientation and manipulation, this configuration allows the same positive positioning aspects described above with regard to the examples of FIGS. 20A and 20B, while permitting such control in a surgical space that offers very little vertical space above the surface of the organ/tissue to be manipulated.

FIG. 20F shows a planar view and FIG. 20G shows a side view of an extremely low profile manipulator 204. Manipulator 204 includes a main body 204a formed of a flexible membrane so that manipulator 204 occupies negligible vertical space above the surface of the organ/tissue to be manipulated. The flexible membrane of main body 204a may be made from silicone, polyurethane, polyester, or other flexible, biocompatible polymer, for example. Grab members 62 may be provided on the superior surface of main body 204a to facilitate positioning and orientation such as by use of one or more graspers in manners as already described above. The inferior surface or contact surface of main body 204a is provided with an adhesive 206 that adheres to the surface of the organ or tissue to be manipulated. The adhesive should be a high-tack, removable, biocompatible adhesive, any of which are known in the art. Once adhered, manipulation may be performed using one or more graspers to grasp one or more grab members 62 and pull, push, rotate, twist or perform other manipulations of the organ/tissue. A non-adhesive layer (not shown) may be provided over the adhesive 206 during insertion and placement/orientation of main body 204a. Once properly positioned and oriented, the non-adhesive layer may be removed to allow adhesion of main body 204a to the surface of the organ/tissue to be manipulated. Main body 204a may be constructed in a wide variety of shapes and sizes. For example, circular configurations, as shown, may be manufactured to have a diameter in the range of about one inch to about 12 inches, or lesser or greater diameters as needed for the particular manipulation to be performed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A stabilizer assembly for stabilizing a portion of an organ in a closed or restricted space surgical site, said assembly comprising:
a stabilizer foot adapted to be delivered to a location of the organ through a first opening in a patient and adapted to contact a surface of the organ to provide stabilization thereto during a surgical procedure; and
a support arm adapted to be delivered to the location of the organ through a second opening in the patient smaller than the first opening;
said stabilizer foot further comprising at least one connecting member and said support arm comprising a connecting feature at a distal end thereof, adapted to connect said support arm to said stabilizer foot via one of said connecting members, said connection being accomplished in the closed or restricted space.

2. The stabilizer assembly of claim 1, wherein said stabilizer foot comprises a plurality of connecting members at varied locations on said stabilizer foot to increase the probability that one of said connecting members will be positioned in a location where said connecting feature is easily connected thereto.

3. The stabilizer assembly of claim 1, wherein said at least one connecting member is relatively rigid.

4. The stabilizer assembly of claim 1, wherein said at least one connecting member is sufficiently malleable to be plastically deformed by hand, but sufficiently rigid so as not to deform during stabilization of the portion of the organ.

5. The stabilizer assembly of claim 1, wherein said at least one connecting member comprises at least one ball member connected to said stabilizer foot via a connecting element.

6. The stabilizer assembly of claim 1, wherein said at least one connecting member comprises at least one slot and recess, and wherein said connecting feature comprises a ball member connected to said distal end of said support arm by a stem, said recess being configured to receive said ball member and said slot being configured to prevent said ball member from passing therethrough.

7. The stabilizer assembly of claim 1, wherein said at least one connecting member comprises an expandable socket member having at least first and second portions, wherein upon compressing said stabilizer foot, said socket member expands, and wherein said socket member contracts to an original configuration upon release of compression of said stabilizer foot.

8. The stabilizer assembly of claim 7, wherein said connecting feature comprises a ball member adapted to be received in said socket member when expanded, and wherein upon contracting to said original configuration, said socket member forms a secure friction fit with said ball member.

9. The stabilizer assembly of claim 1, wherein said stabilizer foot is adapted to apply negative pressure to the surface of the organ, and wherein said at least one connecting member comprises a ball member interconnected with said stabilizer foot via a stem, said ball member and said stem having an opening therethrough adapted to deliver negative pressure to said stabilizer foot.

10. The stabilizer assembly of claim 9, wherein said connecting feature comprises a socket member adapted to mate with said ball member and a seal to provide an airtight seal between said ball member and said support arm.

11. The stabilizer assembly of claim 10, wherein said support arm is adapted to be connected to a vacuum source and to deliver negative pressure to said stabilizer foot through said support arm and said connecting member.

12. The stabilizer assembly of claim 1, wherein said at least one connecting member comprises a first magnetic member and said connecting feature comprises a second magnetic member, said first and second magnetic members interfacing with opposite polarities to draw said support arm into connection with said stabilizer foot.

13. The stabilizer assembly of claim 12, wherein said first and second magnetic members further comprise mating mechanical configurations, wherein upon mating, said mating mechanical configurations provide additional mechanical support to the connection between said stabilizer foot and said support arm.

14. The stabilizer assembly of claim 1, wherein said at least one connecting member comprises a socket member, and wherein said connecting feature comprises an expandable ball member configured to assume a relatively contracted state and a relatively expanded state, wherein when in said relatively contracted state, said expandable ball member is readily positionable within said socket member, and wherein, in said relatively expanded state, said expandable ball member forms a locking connection with said socket member.

15. The stabilizer assembly of claim 14, wherein said expandable ball member comprises a semi-rigid inflatable member.

16. The stabilizer of claim 14, wherein said expandable ball member comprises a split ball member and an interacting wedge member, wherein upon advancing said wedge member into said split ball member, said split ball member expands.

17. The stabilizer assembly of claim 5, wherein said connecting feature comprises jaws adapted to lock onto said ball member.

18. The stabilizer assembly of claim 17, wherein said support arm comprises an outer tube slidable against said jaws to lock said jaws onto said ball member.

19. The stabilizer assembly of claim 17, wherein said jaws are pivotally mounted to said distal end of said support arm.

20. The stabilizer assembly of claim 5, wherein said connecting feature comprises a surface at said distal end of said support arm adapted to contact said ball member, and a finger extending distally of said surface, said finger and said surface at said distal end of said support arm being relatively movable toward one another to capture said ball member.

21. The stabilizer assembly of claim 20, wherein said surface at said distal end of said support arm is drivable toward said finger.

22. The stabilizer assembly of claim 1, wherein said stabilizer foot is adapted to apply negative pressure to the surface of the organ, said assembly further comprising at least one vacuum tube fluidly connected with said stabilizer foot to apply negative pressure thereto when connected, at an opposite end, to a vacuum source, and a yoke slidable over said at least one vacuum tube and connected to said support arm to guide said connecting feature into alignment with said connecting member.

23. The stabilizer assembly of claim 1, further comprising a positioning member adapted to connect with said stabilizer foot for positioning said stabilizer in a desired position and orientation on a surface of the organ, said positioning member being configured to extend from said stabilizer foot, and having sufficient length, while connected to said stabilizer foot and while said stabilizer foot is in contact with the organ, to extend out of said first opening for manipulation by a user.

24. The stabilizer assembly of claim 23, wherein said positioning member is integrally fixed to said stabilizer foot.

25. The stabilizer assembly of claim 23, wherein said positioning member is removable from said stabilizer foot.

26. The stabilizer assembly of claim 25, further comprising a ball joint mechanism formed by interconnection of said positioning member with said stabilizer foot.

27. The stabilizer assembly of claim 25, wherein said positioning member is held directly in contact with said stabilizer foot by a tensioning mechanism.

28. The stabilizer assembly of claim 1, wherein the organ is a beating heart.

29. The stabilizer assembly of claim 1, further comprising at least one grab member on a superior side of said stabilizer foot adapted to be grabbed by a grasping tool, wherein said at least one grab member is adapted to be grasped by extending the grasping tool through the first opening, while controlling the grasping tool from outside the opening.

30. A stabilizer foot for contacting an organ to stabilize a portion thereof in a closed or restricted space surgical site, said stabilizer foot comprising:
at least one contact surface formed on an inferior side of said stabilizer foot adapted to contact a surface of the organ; and
at least one grab member on a superior side of said stabilizer foot adapted to be grabbed by a grasping tool, wherein said stabilizer foot is configured to be inserted through an opening in a patient to contact the surface of the organ, and wherein the at least one grab member is adapted to be grasped by extending the grasping tool through the opening, while controlling the grasping tool from outside the opening.

31. The stabilizer foot of claim 30, comprising a plurality of said grab members distributed about the superior surface of said stabilizer foot to facilitate repositioning said stabilizer foot.

32. The stabilizer foot of claim 31, wherein said grab members comprise partial loops extending from the superior surface of said stabilizer foot.

33. The stabilizer foot of claim 31, wherein said grab members comprise tabs extending from the superior surface of said stabilizer foot.

34. The stabilizer foot of claim 31, wherein said grab members are oriented to facilitate grasping by multiple grasping tools through multiple openings accessing the surgical site.

35. The stabilizer foot of claim 30, further comprising at least one connecting member extending from said stabilizer foot adapted to connect with a support arm having been inserted through a second opening accessing the surgical site.

36. The stabilizer foot of claim 35, wherein said at least one connecting member is relatively rigid.

37. The stabilizer foot of claim 35, wherein said at least one connecting member is sufficiently malleable to be plastically deformed by hand, but sufficiently rigid so as not to deform during stabilization of the portion of the organ.

38. The stabilizer foot of claim 35, wherein said at least one connecting member comprises at least one ball member connected to said stabilizer foot via a connecting element.

39. The stabilizer foot of claim 35, wherein said at least one connecting member comprises at least one slot and recess.

40. The stabilizer foot of claim 35, wherein said at least one connecting member comprises an expandable socket member having at least first and second portions, wherein upon compressing said stabilizer foot, said socket member expands, and wherein said socket member contracts to an original configuration upon release of compression of said stabilizer foot.

41. The stabilizer foot of claim 40, wherein said stabilizer foot comprises a pair of foot members interconnected to form a substantially U-shaped foot member, and wherein said compressing comprises squeezing said foot members toward one another.

42. The stabilizer foot of claim 41, further comprising a weakened section between said foot members to facilitate flexing of said foot members toward one another during said compressing.

43. The stabilizer foot of claim 35, wherein said stabilizer foot is adapted to apply negative pressure to the surface of the organ, and wherein said at least one connecting member comprises a ball member interconnected with said stabilizer foot via a stem, said ball member and said stem having an opening therethrough adapted to deliver negative pressure to said stabilizer foot.

44. The stabilizer foot of claim 35, wherein said at least one connecting member comprises a magnetic member.

45. The stabilizer foot of claim 44, wherein said at least one connecting member further comprises a mechanical configuration adapted to mate with a mating mechanical configuration on a support arm for connecting therewith.

46. The stabilizer foot of claim 35 (Currently Amended) 20., wherein said at least one connecting member comprises a socket member.

47. A stabilizer assembly for stabilizing a portion of an organ in a closed or restricted space surgical site, said assembly comprising:
a stabilizer foot adapted to be delivered to a location of the organ through an opening in a patient and adapted to contact a surface of the organ to provide stabilization thereto during a surgical procedure, said stabilizer foot having at least one opening in a contact surface to apply negative pressure to a surface of the organ, and at least one vacuum line fluidly connected to said at least one contact surface opening and adapted to connect with a vacuum source external of the surgical site; and
soft tissue retracting tapes attached to said stabilizer foot and adapted to be fixed externally of the opening in the patient after connection of said stabilizer foot to the organ via suction to stabilize a portion of the organ.

48. The stabilizer assembly of claim 47, wherein said soft tissue retracting tapes include adhesive on a portion thereof.

49. A stabilizer foot and positioner assembly for use in stabilizing a portion of an organ in a closed or restricted space surgical site, said assembly comprising:
a stabilizer foot adapted to be delivered to a location of the organ through a first opening in a patient and adapted to contact a surface of the organ to provide stabilization thereto during a surgical procedure; and
an elongated positioner extending from said stabilizer foot and having sufficient length to deliver said stabilizer foot to the surface of the organ by manipulating said positioner from outside the first opening.

50. The assembly of claim 49, wherein said positioner is removably connectable to said stabilizer foot.

51. The assembly of claim 50, further comprising a tensioning member having first and second ends, said tensioning member connected to said stabilizer foot at said first end extending through said elongated positioner and having an enlarged anchor at said second end, wherein upon removal of said positioner from connection with said stabilizer foot, said positioner is adapted to slide over said tensioning member and be withdrawn from said first opening, but is incapable of sliding over said enlarged anchor.

52. The assembly of claim 51, wherein said tensioning member further comprises a second anchor intermediate said first and second ends, against which tensioning forces are generated to lock said positioner against said stabilizer foot when said second anchor is engaged with said positioner, and wherein said positioner is configured to slide over said second anchor when said second anchor is not engaged with said positioner.

53. The assembly of claim 52, further comprising a tensioning mechanism for applying tension to said tensioning member to lock said positioner with respect to said stabilizer foot.

54. The assembly of claim 50, further comprising a ball joint interconnection between said positioner and said stabilizer foot.

55. A support arm adapted to connect with a stabilizer foot in a closed or restricted space surgical site, wherein the stabilizer foot has been inserted through a first opening in a patient and the support arm is inserted through a second opening in the patient, said support arm comprising:
an elongated body adapted to pass through the second opening and having sufficient length to extend out of the second opening after connecting said support arm with the stabilizer foot;
a connecting feature disposed at a distal end of said elongated body; and
means for moving said connecting feature between a disconnected configuration in which said connecting feature may be readily disconnected from the stabilizer foot, and a connected configuration, in which said connecting feature securely connects with the stabilizer foot; said means for moving being positioned for manipulation outside of the patient by a user.

56. The support arm of claim 55, wherein said connecting feature comprises jaws.

57. The support arm of claim 55, wherein said connecting feature comprises an expandable ball.

58. The support arm of claim 56, wherein said elongated body comprises an inner shaft member connected to said jaws and an outer tube surrounding said inner shaft member and movable with respect thereto, said means for moving being configured to drive said outer tube against said jaws to move said jaws from said disconnected configuration to said connected configuration.

59. A manipulator assembly for moving or positioning an organ in a closed or restricted space surgical site, said assembly comprising:
a low profile suction member adapted to be delivered to a location of the organ through a first opening in a patient and adapted to contact a surface of the organ and to attach to the organ using vacuum, said suction member further including at least one grab member on a superior surface thereof, said grab members configured to be engaged by a tool for applying force thereto to reposition said suction member on the organ; and
a suction line extending from said suction member and having sufficient length to extend out of said first opening, or a second opening in the patient for connection with an external vacuum source.

60. The manipulator assembly of claim 59, wherein said suction member comprises an elastomeric suction cup and said at least one grab member comprises a plurality of grab members extending from said superior surface of said suction cup.

61. The manipulator assembly of claim 60, wherein said grab members are integrally molded with said suction cup.

62. The manipulator assembly of claim 59, further comprising a positioner having an elongated shaft and a distal end adapted to grasp said grab members, said elongated shaft having sufficient length to enable user manipulation of said distal end, from a proximal end portion thereof located outside the first or second openings, to grasp at least one of said grab members and apply a force thereto to move said suction member.

63. The manipulator assembly of claim 62, wherein said elongated shaft is malleable.

64. The manipulator assembly of claim 59, wherein said at least one grab member comprises a plurality of nubs extending from said superior surface and distributed over said superior surface.

65. The manipulator assembly of claim 59, wherein said at least one grab member comprises a continuous, ring-like projection surrounding said suction member and extending therefrom.

66. The manipulator assembly of claim 59, wherein said suction member further comprises a seal provided on a bottom surface thereof, to enhance establishment of a vacuum seal between said suction member and the surface of the organ.

67. The manipulator assembly of claim 59, wherein said suction member further comprises a diffuser located internally of said suction member to minimize contact of the surface of the organ with one or more openings in said suction member fluidly connected with the suction line.

68. The manipulator assembly of claim 59, wherein said suction member comprises a main body having a superior membrane layer and an inferior membrane layer.

69. The manipulator assembly of claim 68, wherein said inferior membrane layer comprises at least one opening for delivery of vacuum to the surface of the organ.

70. The manipulator assembly of claim 69, further comprising dispersing material placed between said superior membrane layer and said inferior membrane layer.

71. The manipulator assembly of claim 68, further comprising a seal mounted around the perimeter of an inferior surface of said inferior membrane layer.

72. The manipulator assembly of claim 71, further comprising a diffuser adjacent the inferior surface of said inferior membrane, within confines of said seal.

73. The manipulator assembly of claim 59, wherein said suction member comprises a pillow-shaped main body portion.

74. A manipulator assembly for moving or positioning an organ in a closed or restricted space surgical site, said assembly comprising:
a suction member adapted to be delivered to a location of the organ through a first opening in a patient and adapted to contact a surface of the organ and to attach to the organ using vacuum; and
a positioner connected to said suction member, said positioner being flexible in bending and torsionally stiff, said positioner adapted to be drawn through the first opening and rerouted through a second opening in a patient, said positioner having sufficient length to extend proximally out of the second opening when said suction member is attached to a surface of the organ at the location.

75. The manipulator assembly of claim 74, wherein said positioner comprises a coiled layer and a braided layer.

76. The manipulator of claim 74, wherein said positioner comprises first and second coiled layers having opposite directions of winding.

77. The manipulator assembly of claim 74, wherein said positioner is fluidly connected with said suction member, said positioner adapted to be proximally connected to a source of vacuum outside of the patient to deliver vacuum to said suction member inside of the patient.

78. The manipulator assembly of claim 74, further comprising a malleable stylet configured and dimensioned to be inserted through said positioner to conform said positioner to a configuration into which stylet has been shaped prior to insertion into said positioner.

79. The manipulator assembly of claim 78, further comprising a connector attachable to a proximal end of said positioner, said connector including a vacuum port for connecting a source of vacuum thereto, and a second port for introduction of said stylet therethrough.

80. The manipulator assembly of claim 79, wherein said second port includes a seal for making a vacuum-tight seal with said stylet upon introduction of said stylet through said second port.

81. The manipulator assembly of claim 79, wherein said connector comprises a valve that prevents inflow of air through said second port.

82. The manipulator assembly of claim 74, wherein said suction member comprises an elastomeric suction cup.

83. The manipulator assembly of claim 74, wherein said suction member comprises a pillow-shaped main body portion.

84. The manipulator assembly of claim 74, wherein said suction member comprises a main body having a superior membrane layer and an inferior membrane layer.

85. The manipulator assembly of claim 84, wherein said inferior membrane layer comprises at least one opening for delivery of vacuum to the surface of the organ.

86. The manipulator assembly of claim 85, further comprising dispersing material placed between said superior membrane layer and said inferior membrane layer.

87. The manipulator assembly of claim 84, further comprising a seal mounted around the perimeter of an inferior surface of said inferior membrane layer.

88. The manipulator assembly of claim 87, further comprising a diffuser adjacent the inferior surface of said inferior membrane, within confines of said seal.

89. 8& A manipulator assembly for moving or positioning an organ in a closed or restricted space surgical site, said assembly comprising:
a suction member adapted to be delivered to a location of the organ through a first opening in a patient and adapted to contact a surface of the organ and to attach to the organ using vacuum; and
a positioner connected to said suction member, said positioner being flexible in bending and torsionally stiff, said positioner having sufficient length to extend proximally out of the patient when said suction member is attached to a surface of the organ at the location.

90. The manipulator assembly of claim 89, wherein said positioner comprises a coiled layer and a braided layer.

91. The manipulator of claim 89, wherein said positioner comprises first and second coiled layers having opposite directions of winding.

92. A manipulator assembly for moving or positioning an organ in a closed or restricted space surgical site, said assembly comprising:
a low-profile suction member adapted to be delivered to a location of the organ through a first opening in a patient and adapted to contact a surface of the organ and to attach to the organ using vacuum; and
a rigid positioner connected to said suction member, said positioner having sufficient length to extend proximally out of the patient when said suction member is attached to a surface of the organ at the location.

93. The manipulator assembly of claim 92, wherein said suction member comprises a main body having a superior membrane layer and an inferior membrane layer.

94. The manipulator assembly of claim 93, wherein said inferior membrane layer comprises at least one opening for delivery of vacuum to the surface of the organ.

95. The manipulator assembly of claim 94, further comprising dispersing material placed between said superior membrane layer and said inferior membrane layer.

96. The manipulator assembly of claim 93, further comprising a seal mounted around the perimeter of an inferior surface of said inferior membrane layer.

97. The manipulator assembly of claim 96, further comprising a diffuser adjacent the inferior surface of said inferior membrane, within confines of said seal.

98. A suction manipulator comprising:
a suction member adapted to be delivered to a location of an organ through a first opening in a patient and adapted to contact a surface of the organ and to attach to the organ using vacuum; said suction member including at least one grab member on a superior surface thereof, each said grab member configured to be engaged by a tool for applying force thereto to reposition said suction member on the organ.

99. The suction manipulator of claim 98, further comprising a valve adapted to receive an instrument therethrough to establish suction between said suction member and the organ.

100. An extremely low profile manipulator comprising:
a main body formed of a flexible membrane;
at least one grab member on a superior surface of said main body, configured to be engaged by a tool for applying force thereto to reposition said main body on an organ; and
means for attaching said main body to the organ.

101. The manipulator of claim 100, wherein said means for attaching comprises adhesive provided on an inferior surface of said main body.

102. A method of stabilizing a portion of an organ in a closed or restricted space surgical site, said method comprising the steps of:
delivering a stabilizer foot through a first opening in a patient and contacting a surface of the organ with the stabilizer foot;
inserting a distal end of a support arm through a second opening in the patient while controlling the distal end from a proximal end portion of the support arm outside the patient; and
connecting the distal end of the support arm to the stabilizer foot.

103. The method of claim 102, wherein said connecting comprising making a mechanical connection.

104. The method of claim 102, wherein said connecting comprises magnetically connecting.

105. The method of claim 103, wherein said connecting comprises locking jaws provided at said distal end onto a ball member extending from the stabilizer foot.

106. The method of claim 103, wherein said connecting comprises expanding a ball member provided at said distal end against a socket member provided on the stabilizer foot.

107. The method of claim 103 102, wherein said connecting comprises drawing a ball member provided on the stabilizer foot and a socket member at said distal end into contact with one another.

108. The method of claim 102, wherein the stabilizer foot has at least one vacuum line extending therefrom and is adapted to be fixed to the surface of the organ by applying vacuum thereto, said method further comprising:
rerouting the vacuum line by pulling it through the first opening and routing it out of the second opening; and
wherein said connecting comprises threading a yoke component of the support arm over the at least one vacuum line and guiding the distal end of the support arm into position to perform the connection.

109. The method of claim 103, wherein said connecting comprises inserting a ball member provided at said distal end into a recess in the stabilizer foot and drawing the ball member into a slot in the stabilizer foot.

110. The method of claim 102, further comprising fixing the proximal end portion of the support arm to a relatively stationary object outside of the patient.

111. The method of claim 102, further comprising repositioning the stabilizer foot on the surface of the organ prior to said connecting the distal end of the support arm to the stabilizer foot.

112. The method of claim 111, wherein said repositioning comprises moving the stabilizer foot via a connected positioner extending out of the first opening, by applying a force to a proximal portion of the positioner outside of the first opening.

113. The method of claim 112, further comprising disconnecting the positioner from the stabilizer after said connecting the distal end of the support arm to the stabilizer foot; and removing the positioner out of the first opening.

114. The method of claim 113, further comprising capturing the positioner outside of the first opening, so that the positioner maintains attachment to the stabilizer foot via a tensioning member.

115. The method of claim 114, further comprising reconnecting the positioner to the stabilizer foot and repositioning the stabilizer foot again.

116. The method of claim 102, wherein the stabilizer foot is adapted to apply negative pressure to the surface of the organ, said method further comprising applying negative pressure to the surface of the organ to fix the position of stabilizer foot on the organ, prior to said connecting the distal end of the support arm to the stabilizer foot.

117. The method of claim 102, wherein the stabilizer foot is adapted to apply negative pressure to the surface of the organ, said method further comprising applying negative pressure to the surface of the organ to fix the position of stabilizer foot on the organ, after said connecting the distal end of the support arm to the stabilizer foot.

118. The method of claim 111, wherein said repositioning comprises moving the stabilizer foot via a grasping tool extending out of the first opening, by grasping a grab member on the stabilizer foot and applying a force thereto from a proximal portion of the grasping tool outside of the first opening.

119. The method of claim 118, further comprising grasping another grab member on the stabilizer foot to further reposition the stabilizer foot by applying a force to said another grab member via the grasping tool.

120. The method of claim 118, wherein said repositioning further includes moving the stabilizer foot via a second grasping tool extending out of a third opening in the patient, by grasping a grab member on the stabilizer foot and applying a force thereto from a proximal portion of the second grasping tool outside of the third opening.

121. The method of claim 120, wherein one of the grab members is grasped by the second grasping tool before releasing a grasp by the first grasping tool.

122. The method of claim 120, wherein grab members are simultaneously grasped by the first and second grasping tools to triangulate forces on the stabilizer foot to move the stabilizer foot.

123. A method of stabilizing a portion of an organ in a closed or restricted space surgical site, said method comprising the steps of:
delivering a stabilizer foot through a first opening in a patient and contacting a surface of the organ with the stabilizer foot;
applying negative pressure between the stabilizer foot and the organ to fix the stabilizer foot to the organ;
applying tension to soft tissue retractor tapes attached to the stabilizer foot, from a location outside of the first opening, to stabilize a portion of the organ; and
fixing the soft tissue retractor tapes to a relatively stationary object to maintain the applied tension and stabilization.

124. The method of claim 123, wherein said fixing comprises adhering the soft tissue tapes to the skin of the patient outside of the first opening.

125. The method of claim 123, further comprising repositioning the stabilizer foot on the surface of the organ prior to said applying negative pressure.

126. The method of claim 125, wherein said repositioning comprises moving the stabilizer foot via a connected positioner extending out of the first opening, by applying a force to a proximal portion of the positioner outside of the first opening.

127. The method of claim 126, further comprising disconnecting the positioner from the stabilizer after said connecting the distal end of the support arm to the stabilizer foot; and removing the positioner out of the first opening.

128. The method of claim 127, further comprising capturing the positioner outside of the first opening, so that the positioner maintains attachment to the stabilizer foot via a tensioning member.

129. The method of claim 125, wherein said repositioning comprises moving the stabilizer foot via a grasping tool extending out of the first opening, by grasping a grab member on the stabilizer foot and applying a force thereto from a proximal portion of the grasping tool outside of the first opening.

130. The method of claim 129, further comprising grasping another grab member on the stabilizer foot to further reposition the stabilizer foot by applying a force to said another grab member via the grasping tool.

131. The method of claim 129, wherein said repositioning further includes moving the stabilizer foot via a second grasping tool extending out of a second opening in the patient, by grasping a grab member on the stabilizer foot and applying a force thereto from a proximal portion of the second grasping tool outside of the second opening.

132. The method of claim 123, wherein the stabilizer foot has at least one vacuum line extending therefrom through which the negative pressure is applied, said method further comprising:
rerouting the vacuum line by pulling it through the first opening and routing it out of a second opening, prior to connecting the vacuum line to a source of vacuum.

133. A method of positioning a stabilizer on a portion of an organ of a patient in a closed or restricted space surgical site, said method comprising the steps of:
delivering a stabilizer foot through a first opening in a patient and contacting a surface of the organ with the stabilizer foot;
grasping a portion of the stabilizer foot using at least one grasper operated from outside the patient and applying force to the stabilizer foot via the grasper to perform at least one of relocating and reorienting the stabilizer foot; and
removing all graspers from the restricted space surgical site when a desired orientation and location of the stabilizer foot has been achieved.

134. The method of claim 133, wherein at least two graspers are operated to grasp portions of the stabilizer foot during said at least one of orienting and locating, said at least two graspers extending from at least two different openings in the patient.

135. The method of claim 133, further comprising connecting a distal end of a support arm to the stabilizer foot, said support arm having been inserted through a second opening in the patient, wherein said connecting is controlled from a proximal end portion of the support arm outside the patient.

136. A method of manipulating an organ or tissue in a closed or restricted space surgical site, said method comprising the steps of:
delivering a suction member through a first opening in a patient and contacting a surface of the organ or tissue with the suction member;
positioning the suction member in a desired location on the organ or tissue by applying force to the suction member via at least one element extending out of the patient, from a location outside of the patient;
delivering vacuum to the suction member when the suction member has been positioned in the desired location, to establish negative pressure between the suction member and the surface of the organ or tissue such that the suction member attaches to the organ or tissue; and
exerting force on the suction member, via at least one element extending out of the patient, sufficient to move the suction member and the attached organ or tissue.

137. The method of claim 136, wherein said positioning is accomplished by grasping a grab member extending from said suction member and applying force to the grab member.

138. The method of claim 137, wherein said grasping comprises extending a grasper through a second opening in the patient and grasping said grab member.

139. The method of claim 137, wherein said positioning further comprises releasing the grab member and moving the suction member, grasping another grab member and applying force to the another grab member to further move the suction member.

140. The method of claim 137, wherein said positioning comprises grasping grab members with two graspers each of the graspers extending out of different openings through the patient.

141. The method of claim 140, wherein the graspers are applied to alternately grasp and move the suction member.

142. The method of claim 140, wherein the graspers are applied simultaneously to grasp the grab members and move the suction member.

143. The method of claim 136, further comprising drawing a positioner attached to the suction member through the first opening and redirecting the positioner back out through a second opening in the patient.

144. The method of claim 143, wherein said positioning is accomplished by applying a force to the suction member via a proximal portion of the positioner extending out of the patient.

145. The method of claim 144 443, wherein the force comprises pulling on the positioner.

146. The method of claim 144 443, wherein the force comprises torquing the positioner.

147. The method of claim 144, further comprising inserting a stylet within the positioner to stiffen the positioner.

148. The method of claim 147, wherein the force comprises pushing on the stylet and positioner.

149. The method of claim 143, wherein said delivering vacuum comprises delivering vacuum through the positioner.

150. The method of claim 147, wherein said delivering vacuum comprises delivering vacuum through the positioner, and wherein said inserting the stylet is accomplished without interrupting the delivery of vacuum.

151. The method of claim 150, further comprising removing the stylet from the positioner without interrupting the delivery of vacuum.

152. The method of claim 136, further comprising drawing a vacuum tube attached to the suction member through the first opening and redirecting the vacuum tube back out through a second opening in the patient, the vacuum tube having sufficient length to be connected to a source of vacuum outside of the second opening.

153. The method of claim 152, wherein said positioning is accomplished by pulling on a proximal portion of the vacuum tube extending out of the second opening.

154. A method of positioning a manipulator on a portion of an organ or tissue of a patient in a closed or restricted space surgical site, said method comprising the steps of:
delivering a manipulator head through a first opening in a patient and contacting a surface of the organ or tissue with the manipulator head;
grasping a portion of the manipulator head using at least one grasper operated from outside the patient and applying force to the manipulator head via the grasper to perform at least one of relocating and reorienting the manipulator head;
removing all graspers from the restricted space surgical site when a desired orientation and location of the manipulator head has been achieved; and
attaching the manipulator head to the surface of the organ or tissue.

155. The method of claim 154, wherein at least two graspers are operated to grasp portions of the manipulator head during said at least one of orienting and locating, said at least two graspers extending from at least two different openings in the patient.

156. The method of claim 154, wherein the manipulator head comprises a suction member arid wherein said attaching the manipulator head to the surface of the organ or tissue comprises delivering vacuum to the suction member.

157. The method of claim 156, further comprising drawing a vacuum delivery tube attached to the suction member through the first opening and redirecting the vacuum delivery tube back out through a second opening in the patient, and wherein said delivering vacuum comprises connecting a proximal end of the vacuum delivery tube to an external source of vacuum.

158. The method of claim 156, wherein said delivering vacuum comprises inserting an instrument through a valve provided in the suction member, drawing a vacuum inside the suction member using the instrument, and removing the instrument.

159. The method of claim 154, wherein said attaching comprises adhering the manipulator head to the surface of the organ or tissue.

160. A method of manipulating an organ or tissue of a patient in a closed or restricted space surgical site, said method comprising the steps of:
delivering a manipulator head through a first opening in a patient and contacting a surface of the organ or tissue with the manipulator head;
positioning the manipulator head in a desired location on the organ or tissue by grasping a portion of the manipulator head using at least one grasper operated from outside the patient and applying force to the manipulator head via the grasper to perform at least one of relocating and reorienting the manipulator head;
attaching the manipulator head to the surface of the organ or tissue; and
moving the organ or tissue or maintaining the organ or tissue in a displaced location by applying force to at least one portion of the manipulation head via at least one grasper, from at least one location outside of the patient.

161. The method of claim 160, wherein the manipulator head comprises a suction member and wherein said attaching comprises delivering vacuum to the suction member.

162. The method of claim 160, wherein the manipulator head comprises a flexible membrane, and wherein said attaching comprises adhering the flexible membrane to the surface of the organ or tissue.
